SEQUENCE LISTING (9 sequences)

SEQ ID NO: 1 - protein, 832 aa, unidentified
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLCCCDPNLQNIPVRTPLGQRIRRGFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 2 - protein, 553 aa, synthetic construct
MLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 3 - protein, 553 aa, synthetic construct
MLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWCLVALDYSQIELRVLAHLSGDENLIRVFQEGRDIHTETASWMFGVPREAVDPLMRRAAKIINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 4 - protein, 553 aa, synthetic construct
MLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKIINFGVLYGMSAHRLSQELAIPYEEAQAFIERYFQSFPKVRAWIEKTLEEGRRRGYVETLFGRRRYVPALEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 5 - protein, 553 aa, synthetic construct
MLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKIINFGVLYGMSAHRLSQELAIPYEEAQAFIERYFQSFPKVRAWIEKTLEEGRRRGYVETLFGRRRYVPDLEAQVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 6 - protein, 553 aa, synthetic construct
MLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERLLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKIINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 7 - protein, 553 aa, synthetic construct
MLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQGLAIPYEEAQAFIERYFQSFPKVRAYIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIAEDWLSAKE

SEQ ID NO: 8 - protein, 553 aa, synthetic construct
MLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGFRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKIINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 9 - protein, 553 aa, synthetic construct
MLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEAIREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEAKVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

(19) United States
(12) Patent Application Publication
(10) Pub. No.: US 2026/0308540 A1
(43) Pub. Date: Oct. 8, 2026

(54) POLYMERASE VARIANTS

(71) Applicant: Twist Bioscience Corporation, South San Francisco, CA (US)

(72) Inventors: Owen Kabnick SMITH, Mountain View, CA (US); Sean Patrick TIGHE, Soquel, CA (US); Xuan Yu Elian LEE, San Mateo, CA (US); Ramsey Ibrahim ZEITOUN, Westminster, CO (US); Siyuan CHEN, Palo Alto, CA (US)

(73) Assignee: Twist Bioscience Corporation, South San Francisco, CA (US)

(21) Appl. No.: 19/477,295

(22) PCT Filed: Apr. 17, 2024
(86) PCT No.: PCT/US2024/024895

§ 371 Date: Oct. 21, 2025

Related U.S. Application Data
(60) Provisional application No. 63/497,665, filed on Apr. 21, 2023.

(51) Int. Cl. C12N 9/12; C12N 15/10; C12P 19/34; C12Q 1/6806; C12Q 1/6855

(52) U.S. Cl. CPC C12N 9/1252; C12N 15/1093; C12P 19/34; C12Q 1/6806; C12Q 1/6855; C07K 2319/21; C12Y 207/07007

(57) ABSTRACT

Described herein are methods and compositions relating to enzyme polypeptides and libraries having nucleic acids encoding for the polypeptides comprising variant amino acid sequences. Further described herein are methods of extending polynucleotide molecules using enzyme polypeptides having variant amino acid sequences. Further described herein are methods for preparing sequencing libraries using polymerase polypeptides having variant amino acid sequences.

### CROSS-REFERENCE TO RELATED APPLICATIONS

This application claims priority to U.S. Provisional Patent Application No. 63/497,665 filed Apr. 21, 2023, the entirety of which is incorporated herein by reference. All publications, patents, and patent applications mentioned in this specification are herein incorporated by reference to the same extent as if each individual publication, patent, or patent application was specifically and individually indicated to be incorporated by reference.

### BACKGROUND

Enzymes are capable of catalyzing a wide range of chemical reactions, including those used in chemical biology for sequencing applications. However, the design and implementation of enzymes can be challenging. Thus, there is a need to develop compositions and methods for the optimization of enzyme properties.

### SUMMARY

Provided herein are polypeptides comprising amino acid sequences comprising at least one amino acid mutation relative to SEQ ID NO: 1. In some embodiments, the amino acid sequence at least 80%, at least 90%, at least 95%, at least 98%, or 100% homologous to any one of SEQ ID NOs: 3-9. In some embodiments, the mutation comprises an addition, deletion, substitution, or combination thereof. In some embodiments, the deletion comprises 250-300 amino acids from the N-terminus relative to SEQ ID NO: 1. In some embodiments, the polypeptide comprises at least 2, at least 3, or at least 4 amino acid mutations relative to SEQ ID NO: 1. In some embodiments, the mutations are at one or more of positions V449, V493, L522, L605, T664, E681, W706, D732, R736, R736, and G824 relative to SEQ ID NO: 1. In some embodiments, the mutations are selected from one or more of V449F, V493L, L522I, L605C, T664I, E681G, W706Y, D732A, R736K, R736Q, and G824A relative to SEQ ID NO: 1. In some embodiments, the polypeptide comprises a purification tag.

Also provided herein are nucleic acid molecules encoding for the polypeptides, and vectors and cells comprising the nucleic acid molecules.

Provided herein are methods for extending a first polynucleotide. In some aspects, the method comprises contacting the first polynucleotide with a nucleotide and a polypeptide to form an extended polynucleotide. In some aspects, the polypeptide comprises an amino acid sequence comprising at least one amino acid mutation relative to SEQ ID NO: 1. In some embodiments, the first polynucleotide comprises genomic DNA or a fragment thereof, cDNA, or adenosine triphosphate. In some embodiments, the method is at least 90% selective for incorporation of a single nucleotide. In some embodiments, the method is at least 90% selective for incorporation of a nucleotide type. In some embodiments, the method is at least 95% selective for adenine (A) over guanine (G). In some embodiments, the method further comprises ligating an adapter to the extended polynucleotide. In some embodiments, the adapter comprises a complementary overhang to the extended polynucleotide. In some embodiments, the method further comprises extending a second polynucleotide. In some aspects, the polynucleotide and the second polynucleotide are hybridized.

Provided herein are methods for preparing a sequencing library. In some aspects, method comprises providing a plurality of nucleic acids; end-repairing the plurality of nucleic acids; performing a-tailing on the plurality of nucleic acids using a polymerase; and ligating at least one adapter to the nucleic acids using a ligase. In some aspects, the polymerase comprises an amino acid sequence comprising at least one amino acid mutation relative to SEQ ID NO: 1.

### BRIEF DESCRIPTION OF THE DRAWINGS

FIG. 1 is a diagram depicting an exemplary workflow for assaying A-tailing activity of variants of TaqIT DNA polymerase (“TaqIT”), according to aspects of the present disclosure.

FIGS. 2A-2D is a bar graph demonstrating end compositions of an exemplary adapter before and after end-repairing and A-tailing, according to aspects of the present disclosure. FIG. 2A depicts read counts for untreated cell-free DNA (cfDNA) molecules having blunt ends or overhangs of varying lengths. FIG. 2B depicts read counts for end-repaired cfDNA molecules having blunt ends or overhangs of varying lengths. FIG. 2C depicts read counts for end-repaired and A-tailed cfDNA molecules having blunt ends or overhangs of varying lengths. FIG. 2D depicts an end composition of one base pair having a 3′ overhang added by wild-type TaqIT DNA polymerase.

FIG. 3 is a probability plot depicting cumulative probabilities for amino acids (0.0 to 1.0 at 0.2 unit intervals) versus position in Taq DNA polymerase (left to right: 730-755), according to aspects of the present disclosure.

FIG. 4A is a scatter plot depicting mean-normalized results from an exemplary first round screen of A-tailing variants of DNA polymerase, according to aspects of the present disclosure. FIG. 4B is a table depicting fold change values of top performer variants over wild-type DNA polymerase, according to aspects of the present disclosure.

FIGS. 5A-5C demonstrate results of an exemplary experiment comparing n Taq DNA polymerase homologues to wild-type, according to aspects of the present disclosure. FIG. 5A is a photograph of an SDS-PAGE gel of two purified wild-type DNA polymerases. FIG. 5B is a photograph of an SDS-PAGE gel of twelve purified homologues of Taq DNA polymerases. FIG. 5C is a bar graph depicting results of next-generation sequencing performed with each of the twelve Taq DNA polymerase homologues and the two wild-type DNA polymerases.

FIGS. 6A-6C depict results of an exemplary experiment comparing binary A-tailing variants of TaqIT DNA polymerase to wild-type, according to aspects of the present disclosure. FIG. 6A is a scatter plot depicting normalized results from exemplary binary A-tailing variants of TaqIT DNA Polymerase.

FIG. 6B is a table depicting fold change value of top performer binary variants over wild-type. FIG. 6C is a scatter plot depicting additional results from binary A-tailing variants of TaqIT DNA polymerase.

FIGS. 7A-7C depicts results of an exemplary experiment evaluating binary variants of TaqIT DNA polymerase, according to aspects of the present disclosure. FIG. 7A is a photograph of an SDS-PAGE gel of purified binary variants of TaqIT DNA polymerase. FIG. 7B is a bar graph depicting results from next-generation sequencing performed with binary variants as compared to wild-type. FIG. 7C is a bar graph depicting additional binary variants next-generation sequencing results.

FIGS. 8A-8B depict results of an exemplary experiment evaluating effectiveness of binary A-tailing variants of TaqIT DNA polymerase, according to aspects of the present disclosure. FIG. 8A is a bar graph depicting fraction reads with correct tail length after A-tailing with the binary variants. FIG. 8B is a bar graph depicting fraction reads of single-base pair 3′ overhangs that had a guanine (G) instead of an adenosine (A) addition.

FIGS. 9A-9B depicts results of an exemplary experiment evaluating tertiary variants of TaqIT DNA polymerase, according to aspects of the present disclosure. FIG. 9A is a scatter plot depicting normalized results from the tertiary variants. FIG. 9B is a table depicting fold change values of top performer tertiary variants over wild-type.

### DETAILED DESCRIPTION

The present disclosure employs, unless otherwise indicated, conventional molecular biology techniques, which are within the skill of the art. Unless defined otherwise, all technical and scientific terms used herein have the same meaning as is commonly understood by one of ordinary skill in the art.

Provided herein are compositions and methods for generation of sequencing libraries. Further provided herein are engineered enzymes to improve library generation. Further provided herein are polymerases for generating sequencing libraries.

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

Unless specifically stated otherwise, as used herein, the terms “nucleic acid”, “nucleic acid molecule”, “polynucleotide”, and “oligonucleotide” encompass double-stranded or triple-stranded nucleic acid molecules, as well as single-stranded nucleic acid molecules. In double-stranded or triple-stranded nucleic acid molecules, the nucleic acid strands need not be coextensive (i.e., a double-stranded nucleic acid molecule need not be double-stranded along the entire length of both strands). Nucleic acid sequences, when provided, are listed in the 5′ to 3′ direction, unless stated otherwise. Methods described herein provide for the generation of isolated nucleic acids. Methods described herein additionally provide for the generation of isolated and purified nucleic acids. A “nucleic acid” as referred to herein can comprise at least 5, 10, 20, 30, 40, 50, 60, 70, 80, 90, 100, 125, 150, 175, 200, 225, 250, 275, 300, 325, 350, 375, 400, 425, 450, 475, 500, 600, 700, 800, 900, 1000, 1100, 1200, 1300, 1400, 1500, 1600, 1700, 1800, 1900, 2000, or more bases in length. Moreover, provided herein are methods for the synthesis of any number of polypeptide-segments encoding nucleotide sequences, including sequences encoding non-ribosomal peptides (NRPs), sequences encoding non-ribosomal peptide-synthetase (NRPS) modules and synthetic variants, polypeptide segments of other modular proteins, such as antibodies, polypeptide segments from other protein families, including non-coding DNA or RNA, such as regulatory sequences e.g. promoters, transcription factors, enhancers, siRNA, shRNA, RNAi, miRNA, small nucleolar RNA derived from microRNA, or any functional or structural DNA or RNA unit of interest.

The following are non-limiting examples of polynucleotides: coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, complementary DNA (cDNA), which is a DNA representation of mRNA, usually obtained by reverse transcription of messenger RNA (mRNA) or by amplification; DNA molecules produced synthetically or by amplification, genomic DNA, recombinant polynucleotides, branched polynucleotides, plasmids, vectors, isolated DNA of any sequence, isolated RNA of any sequence, nucleic acid probes, and primers. cDNA encoding for a gene or gene fragment referred herein may comprise at least one region encoding for exon sequences without an intervening intron sequence in the genomic equivalent sequence.

### Enzyme Variants

Provided herein are enzymes for library preparation. In some instances, the enzyme comprises a polymerase. In some instances, the enzyme is configured to increase the specificity of non-templated 3′ nucleotide addition. In some instances, the enzyme is configured to increase the specificity of non-templated 3′ adenosine addition. In some instances, the enzyme comprise a Taq polymerase. In some instances, the Taq polymerase is selected from Table 1, below. In some instances, the Taq polymerase is a truncated Taq polymerase (e.g., a TaqIT polymerase). In some instances, the enzyme comprises a variant of SEQ ID NO: 1. In some instances, the enzyme comprises a variant of SEQ ID NO: 2.

Taq polymerases may be used for “A-tailing”, wherein an adenosine nucleotide is extended from the 3′ end of a polynucleotide (e.g., genomic DNA, cDNA). In some instances, extension to generate an overhang facilitates ligation with adapters. In some instances, ligation occurs using T4 ligase or other ligase. In some instances, variant polymerases provided herein provide for higher control over the number of nucleotides added. In some instances, the nucleotide comprises adenosine triphosphate. In some instances, the variant enzyme comprises at least 70%, 75%, 80%, 85%, 90%, 95%, 97%, or at least 99% selectivity for a single nucleotide vs. incorporation of multiple nucleotides (e.g., 2 or more). Variant polymerases provided herein in some instances provide for higher control over the type of nucleotides added. In some instances, the variant polymerase comprises at least 70%, 75%, 80%, 85%, 90%, 95%, 97%, or at least 99% selectivity for a single nucleotide type. In some instances, the variant polymerase comprises at least 70%, 75%, 80%, 85%, 90%, 95%, 97%, or at least 99% selectivity for adenosine. In some instances, the variant polymerase comprises at least 70%, 75%, 80%, 85%, 90%, 95%, 97%, or at least 99% selectivity for a single nucleotide type for adenosine (A) over guanosine (G). In some instances, variant polymerase extend the 3′ end of a first polynucleotide and a second polynucleotide. In some instances, a first polynucleotide and a second polynucleotide are hybridized together.

An enzyme provided herein may comprise one or more variants of SEQ ID NO: 1. In some instances, a variant comprises one or more of an insertion, deletion, or substitution relative to SEQ ID NO: 1. In some instances, a deletion comprises an N-terminal deletion. In some instances, a deletion comprises a C-terminal deletion. In some instances, a deletion comprises a deletion of at least 10, 25, 30, 50, 60, 100, 150, 200, 250, 280, 300, or at least 350 amino acids. In some instances, a deletion comprises a deletion of at least 10, 25, 30, 50, 60, 100, 150, 200, 250, 280, 300, or at least 350 amino acids from the N-terminus. In some instances, a deletion comprises a deletion of 20-300, 20-290, 20-250, 20-200, 50-300, 100-300, 150-300, 200-300, 200-350, 200-400, 250-400, 250-300, 250-350, 275-300, or 275-325 amino acids. In some instances, a deletion comprises a deletion of 20-300, 20-290, 20-250, 20-200, 50-300, 100-300, 150-300, 200-300, 200-350, 200-400, 250-400, 250-300, 250-350, 275-300, or 275-325 amino acids from the N-terminus. In some instances, a variant comprises at least 1, at least 2, at least 3, at least 4, at least 5, at least 6, at least 7, at least 8, at least 9, at least 10, at least 11, at least 12, at least 13, at least 14, at least 15, or at least 16 variant amino acid positions of SEQ ID NO: 1. In some instances, a variant comprises about 1, about 2, about 3, about 4, about 5, about 6, about 7, about 8, about 9, about 10, about 11, about 12, about 13, about 14, about 15, or about 16 variant amino acid positions of SEQ ID NO: 1. An enzyme provided herein may comprise one or more variants of SEQ ID NO: 2. In some instances, a variant comprises at least 1, at least 2, at least 3, at least 4, at least 5, at least 6, at least 7, at least 8, at least 9, at least 10, at least 11, at least 12, at least 13, at least 14, at least 15, or at least 16 variant amino acid positions of SEQ ID NO: 2. In some instances, a variant comprises about 1, about 2, about 3, about 4, about 5, about 6, about 7, about 8, about 9, about 10, about 11, about 12, about 13, about 14, about 15, or about 16 variant amino acid positions of SEQ ID NO: 2.

An enzyme provided herein may comprise a sequence having homology or similarity and mutations at one or more amino acid positions. In some instances, an enzyme comprises a mutation at one or more of positions selected from 449, 493, 522, 605, 664, 681, 706, 732, 736, or 824 and at least 95% similarity to SEQ ID NO: 1. In some instances, an enzyme comprises a mutation at two or more of positions selected from 449, 493, 522, 605, 664, 681, 706, 732, 736, or 824 and at least 90% similarity to SEQ ID NO: 1. In some instances, an enzyme comprises a mutation at three or more of positions selected from 449, 493, 522, 605, 664, 681, 706, 732, 736, or 824 and at least 90% similarity to SEQ ID NO: 1. In some instances, an enzyme comprises a mutation at four or more of positions selected from 449, 493, 522, 605, 664, 681, 706, 732, 736, or 824 and at least 90% similarity to SEQ ID NO: 1. In some instances, an enzyme comprises a mutation at five or more of positions selected from 449, 493, 522, 605, 664, 681, 706, 732, 736, or 824 and at least 90% similarity to SEQ ID NO: 1. In some instances, an enzyme comprises a mutation at six or more of positions selected from 449, 493, 522, 605, 664, 681, 706, 732, 736, or 824 and at least 90% similarity to SEQ ID NO: 1. In some instances, an enzyme comprises a mutation at seven or more of positions selected from 449, 493, 522, 605, 664, 681, 706, 732, 736, or 824 and at least 90% similarity to SEQ ID NO: 1. In some instances, an enzyme comprises a mutation at eight or more of positions selected from 449, 493, 522, 605, 664, 681, 706, 732, 736, or 824 and at least 90% similarity to SEQ ID NO: 1. In some instances, an enzyme comprises a mutation at nine or more of positions selected from 449, 493, 522, 605, 664, 681, 706, 732, 736, or 824 and at least 90% similarity to SEQ ID NO: 1. In some instances, an enzyme comprises a mutation at ten or more of positions selected from 449, 493, 522, 605, 664, 681, 706, 732, 736, or 824 and at least 90% similarity to SEQ ID NO: 1.

An enzyme provided herein may comprise a sequence having homology or similarity and mutations at one or more amino acid positions. In some instances, an enzyme comprises a mutation at one or more of positions selected from V449F, V493L, L522I, L605C, T664I, E681G, W706Y, D732A, R736K, R736Q, or G824A and at least 95% similarity to SEQ ID NO: 1. In some instances, an enzyme comprises a mutation at two or more of positions selected from V449F, V493L, L522I, L605C, T664I, E681G, W706Y, D732A, R736K, R736Q, or G824A and at least 90% similarity to SEQ ID NO: 1. In some instances, an enzyme comprises a mutation at three or more of positions selected from V449F, V493L, L522I, L605C, T664I, E681G, W706Y, D732A, R736K, R736Q, or G824A and at least 90% similarity to SEQ ID NO: 1. In some instances, an enzyme comprises a mutation at four or more of positions selected from V449F, V493L, L522I, L605C, T664I, E681G, W706Y, D732A, R736K, R736Q, or G824A and at least 90% similarity to SEQ ID NO: 1. In some instances, an enzyme comprises a mutation at five or more of positions selected from V449F, V493L, L522I, L605C, T664I, E681G, W706Y, D732A, R736K, R736Q, or G824A and at least 90% similarity to SEQ ID NO: 1. In some instances, an enzyme comprises a mutation at six or more of positions selected from V449F, V493L, L522I, L605C, T664I, E681G, W706Y, D732A, R736K, R736Q, or G824A and at least 90% similarity to SEQ ID NO: 1. In some instances, an enzyme comprises a mutation at seven or more of positions selected from V449F, V493L, L522I, L605C, T664I, E681G, W706Y, D732A, R736K, R736Q, or G824A and at least 90% similarity to SEQ ID NO: 1. In some instances, an enzyme comprises a mutation at eight or more of positions selected from V449F, V493L, L522I, L605C, T664I, E681G, W706Y, D732A, R736K, R736Q, or G824A and at least 90% similarity to SEQ ID NO: 1. In some instances, an enzyme comprises a mutation at nine or more of positions selected from V449F, V493L, L522I, L605C, T664I, E681G, W706Y, D732A, R736K, R736Q, or G824A and at least 90% similarity to SEQ ID NO: 1. In some instances, an enzyme comprises a mutation at ten or more of positions selected from V449F, V493L, L522I, L605C, T664I, E681G, W706Y, D732A, R736K, R736Q, or G824A and at least 90% similarity to SEQ ID NO: 1.

An enzyme provided herein may comprise one or more variants of SEQ ID NO: 2. In some instances, a variant comprises at least 1, at least 2, at least 3, at least 4, at least 5, at least 6, at least 7, at least 8, at least 9, at least 10, at least 11, at least 12, at least 13, at least 14, at least 15, or at least 16 variant amino acid positions of SEQ ID NO: 2. In some instances, a variant comprises about 1, about 2, about 3, about 4, about 5, about 6, about 7, about 8, about 9, about 10, about 11, about 12, about 13, about 14, about 15, or about 16 variant amino acid positions of SEQ ID NO: 2.

An enzyme provided herein may comprise a sequence having homology or similarity and mutations at one or more amino acid positions. In some instances, an enzyme comprises a mutation at one or more of positions selected from 449, 493, 522, 605, 664, 681, 706, 732, 736, or 824 and at least 95% similarity to SEQ ID NO: 1. In some instances, an enzyme comprises a mutation at two or more of positions selected from 449, 493, 522, 605, 664, 681, 706, 732, 736, or 824 and at least 90% similarity to SEQ ID NO: 1. In some instances, an enzyme comprises a mutation at three or more of positions selected from 449, 493, 522, 605, 664, 681, 706, 732, 736, or 824 and at least 90% similarity to SEQ ID NO: 1. In some instances, an enzyme comprises a mutation at four or more of positions selected from 449, 493, 522, 605, 664, 681, 706, 732, 736, or 824 and at least 90% similarity to SEQ ID NO: 1. In some instances, an enzyme comprises a mutation at five or more of positions selected from 449, 493, 522, 605, 664, 681, 706, 732, 736, or 824 and at least 90% similarity to SEQ ID NO: 1. In some instances, an enzyme comprises a mutation at six or more of positions selected from 449, 493, 522, 605, 664, 681, 706, 732, 736, or 824 and at least 90% similarity to SEQ ID NO: 1. In some instances, an enzyme comprises a mutation at seven or more of positions selected from 449, 493, 522, 605, 664, 681, 706, 732, 736, or 824 and at least 90% similarity to SEQ ID NO: 1. In some instances, an enzyme comprises a mutation at eight or more of positions selected from 449, 493, 522, 605, 664, 681, 706, 732, 736, or 824 and at least 90% similarity to SEQ ID NO: 1. In some instances, an enzyme comprises a mutation at nine or more of positions selected from 449, 493, 522, 605, 664, 681, 706, 732, 736, or 824 and at least 90% similarity to SEQ ID NO: 1. In some instances, an enzyme comprises a mutation at ten or more of positions selected from 449, 493, 522, 605, 664, 681, 706, 732, 736, or 824 and at least 90% similarity to SEQ ID NO: 1.

An enzyme provided herein may comprise a sequence having homology or similarity and mutations at one or more amino acid positions. In some instances, an enzyme comprises a mutation at one or more of positions selected from V449F, V493L, L522I, L605C, T664I, E681G, W706Y, D732A, R736K, R736Q, or G824A and at least 95% similarity to SEQ ID NO: 2. In some instances, an enzyme comprises a mutation at two or more of positions selected from V449F, V493L, L522I, L605C, T664I, E681G, W706Y, D732A, R736K, R736Q, or G824A and at least 90% similarity to SEQ ID NO: 2. In some instances, an enzyme comprises a mutation at three or more of positions selected from V449F, V493L, L522I, L605C, T664I, E681G, W706Y, D732A, R736K, R736Q, or G824A and at least 90% similarity to SEQ ID NO: 2. In some instances, an enzyme comprises a mutation at four or more of positions selected from V449F, V493L, L522I, L605C, T664I, E681G, W706Y, D732A, R736K, R736Q, or G824A and at least 90% similarity to SEQ ID NO: 2. In some instances, an enzyme comprises a mutation at five or more of positions selected from V449F, V493L, L522I, L605C, T664I, E681G, W706Y, D732A, R736K, R736Q, or G824A and at least 90% similarity to SEQ ID NO: 2. In some instances, an enzyme comprises a mutation at six or more of positions selected from V449F, V493L, L522I, L605C, T664I, E681G, W706Y, D732A, R736K, R736Q, or G824A and at least 90% similarity to SEQ ID NO: 2. In some instances, an enzyme comprises a mutation at seven or more of positions selected from V449F, V493L, L522I, L605C, T664I, E681G, W706Y, D732A, R736K, R736Q, or G824A and at least 90% similarity to SEQ ID NO: 2. In some instances, an enzyme comprises a mutation at eight or more of positions selected from V449F, V493L, L522I, L605C, T664I, E681G, W706Y, D732A, R736K, R736Q, or G824A and at least 90% similarity to SEQ ID NO: 2. In some instances, an enzyme comprises a mutation at nine or more of positions selected from V449F, V493L, L522I, L605C, T664I, E681G, W706Y, D732A, R736K, R736Q, or G824A and at least 90% similarity to SEQ ID NO: 2. In some instances, an enzyme comprises a mutation at ten or more of positions selected from V449F, V493L, L5221, L605C, T664I, E681G, W706Y, D732A, R736K, R736Q, or G824A and at least 90% similarity to SEQ ID NO: 2.

All sequences were in some instances expressed with a His6 tag (HHHHHH, SEQ ID NO: 10) for purification purposes at the C-terminus of the polypeptide sequence.

TABLE 1
Polymerase Protein Sequences

| SEQ ID NO | Name | Protein Sequence |
|---|---|---|
| 1 | WT_Taq_Polymerase | MRGMLPLFEPKGRVLLVDGHHLAYRTFHALKGLTTSRG |
| | | EPVQAVYGFAKSLLKALKEDGDAVIVVFDAKAPSFRHE |
| | | AYGGYKAGRAPTPEDFPRQLALIKELVDLLGLARLEVP |
| | | GYEADDVLASLAKKAEKEGYEVRILTADKDLYQLLSDR |
| | | IHVLHPEGYLITPAWLWEKYGLRPDQWADYRALTGDES |
| | | DNLPGVKGIGEKTARKLLEEWGSLEALLKNLDRLKPAI |
| | | REKILAHMDDLKLSWDLAKVRTDLPLEVDFAKRREPDR |
| | | ERLRAFLERLEFGSLLHEFGLLESPKALEEAPWPPPEG |
| | | AFVGFVLSRKEPMWADLLALAAARGGRVHRAPEPYKAL |
| | | RDLKEARGLLAKDLSVLALREGLGLPPGDDPMLLAYLL |
| | | DPSNTTPEGVARRYGGEWTEEAGERAALSERLFANLWG |
| | | RLEGEERLLWLYREVERPLSAVLAHMEATGVRLDVAYL |
| | | RALSLEVAEEIARLEAEVFRLAGHPFNLNSRDQLERVL |
| | | FDELGLPAIGKTEKTGKRSTSAAVLEALREAHPIVEKI |
| | | LQYRELTKLKSTYIDPLPDLIHPRTGRLHTRFNQTATA |
| | | TGRLCCCDPNLQNIPVRTPLGQRIRRGFIAEEGWLLVA |
| | | LDYSQIELRVLAHLSGDENLIRVFQEGRDIHTETASWM |
| | | FGVPREAVDPLMRRAAKTINFGVLYGMSAHRLSQELAI |
| | | PYEEAQAFIERYFQSFPKVRAWIEKTLEEGRRRGYVET |
| | | LFGRRRYVPDLEARVKSVREAAERMAFNMPVQGTAADL |
| | | MKLAMVKLFPRLEEMGARMLLQVHDELVLEAPKERAEA |
| | | VARLAKEVMEGVYPLAVPLEVEVGIGEDWLSAKE |
| 2 | TaqIT_Polymerase | MLLHEFGLLESPKALEEAPWPPPEGAFVGFVLSRKEPM |
| | | WADLLALAAARGGRVHRAPEPYKALRDLKEARGLLAKD |
| | | LSVLALREGLGLPPGDDPMLLAYLLDPSNTTPEGVARR |
| | | YGGEWTEEAGERAALSERLFANLWGRLEGEERLLWLYR |
| | | EVERPLSAVLAHMEATGVRLDVAYLRALSLEVAEEIAR |
| | | LEAEVFRLAGHPFNLNSRDQLERVLFDELGLPAIGKTE |
| | | KTGKRSTSAAVLEALREAHPIVEKILQYRELTKLKSTY |
| | | IDPLPDLIHPRTGRLHTRFNQTATATGRLSSSDPNLQN |
| | | IPVRTPLGQRIRRAFIAEEGWLLVALDYSQIELRVLAH |
| | | LSGDENLIRVFQEGRDIHTETASWMFGVPREAVDPLMR |
| | | RAAKTINFGVLYGMSAHRLSQELAIPYEEAQAFIERYF |
| | | QSFPKVRAWIEKTLEEGRRRGYVETLFGRRRYVPDLEA |
| | | RVKSVREAAERMAFNMPVQGTAADLMKLAMVKLFPRLE |
| | | EMGARMLLQVHDELVLEAPKERAEAVARLAKEVMEGVY |
| | | PLAVPLEVEVGIGEDWLSAKE |
| 3 | TaqIT_L605C_T664I | MLLHEFGLLESPKALEEAPWPPPEGAFVGFVLSRKEPM |
| | | WADLLALAAARGGRVHRAPEPYKALRDLKEARGLLAKD |
| | | LSVLALREGLGLPPGDDPMLLAYLLDPSNTTPEGVARR |
| | | YGGEWTEEAGERAALSERLFANLWGRLEGEERLLWLYR |
| | | EVERPLSAVLAHMEATGVRLDVAYLRALSLEVAEEIAR |
| | | LEAEVFRLAGHPFNLNSRDQLERVLFDELGLPAIGKTE |
| | | KTGKRSTSAAVLEALREAHPIVEKILQYRELTKLKSTY |
| | | IDPLPDLIHPRTGRLHTRFNQTATATGRLSSSDPNLQN |
| | | IPVRTPLGQRIRRAFIAEEGWCLVALDYSQIELRVLAH |
| | | LSGDENLIRVFQEGRDIHTETASWMFGVPREAVDPLMR |
| | | RAAKIINFGVLYGMSAHRLSQELAIPYEEAQAFIERYF |
| | | QSFPKVRAWIEKTLEEGRRRGYVETLFGRRRYVPDLEA |
| | | RVKSVREAAERMAFNMPVQGTAADLMKLAMVKLFPRLE |
| | | EMGARMLLQVHDELVLEAPKERAEAVARLAKEVMEGVY |
| | | PLAVPLEVEVGIGEDWLSAKE |
| 4 | TaqIT_T664I_D732A | MLLHEFGLLESPKALEEAPWPPPEGAFVGFVLSRKEPM |
| | | WADLLALAAARGGRVHRAPEPYKALRDLKEARGLLAKD |
| | | LSVLALREGLGLPPGDDPMLLAYLLDPSNTTPEGVARR |
| | | YGGEWTEEAGERAALSERLFANLWGRLEGEERLLWLYR |
| | | EVERPLSAVLAHMEATGVRLDVAYLRALSLEVAEEIAR |
| | | LEAEVFRLAGHPFNLNSRDQLERVLFDELGLPAIGKTE |
| | | KTGKRSTSAAVLEALREAHPIVEKILQYRELTKLKSTY |
| | | IDPLPDLIHPRTGRLHTRFNQTATATGRLSSSDPNLQN |
| | | IPVRTPLGQRIRRAFIAEEGWLLVALDYSQIELRVLAH |
| | | LSGDENLIRVFQEGRDIHTETASWMFGVPREAVDPLMR |
| | | RAAKIINFGVLYGMSAHRLSQELAIPYEEAQAFIERYF |
| | | QSFPKVRAWIEKTLEEGRRRGYVETLFGRRRYVPALEA |
| | | RVKSVREAAERMAFNMPVQGTAADLMKLAMVKLFPRLE |
| | | EMGARMLLQVHDELVLEAPKERAEAVARLAKEVMEGVY |
| | | PLAVPLEVEVGIGEDWLSAKE |
| 5 | TaqIT_T664I_R736Q | MLLHEFGLLESPKALEEAPWPPPEGAFVGFVLSRKEPM |
| | | WADLLALAAARGGRVHRAPEPYKALRDLKEARGLLAKD |
| | | LSVLALREGLGLPPGDDPMLLAYLLDPSNTTPEGVARR |
| | | YGGEWTEEAGERAALSERLFANLWGRLEGEERLLWLYR |
| | | EVERPLSAVLAHMEATGVRLDVAYLRALSLEVAEEIAR |
| | | LEAEVFRLAGHPFNLNSRDQLERVLFDELGLPAIGKTE |
| | | KTGKRSTSAAVLEALREAHPIVEKILQYRELTKLKSTY |
| | | IDPLPDLIHPRTGRLHTRFNQTATATGRLSSSDPNLQN |
| | | IPVRTPLGQRIRRAFIAEEGWLLVALDYSQIELRVLAH |
| | | LSGDENLIRVFQEGRDIHTETASWMFGVPREAVDPLMR |
| | | RAAKIINFGVLYGMSAHRLSQELAIPYEEAQAFIERYF |
| | | QSFPKVRAWIEKTLEEGRRRGYVETLFGRRRYVPDLEA |
| | | QVKSVREAAERMAFNMPVQGTAADLMKLAMVKLFPRLE |
| | | EMGARMLLQVHDELVLEAPKERAEAVARLAKEVMEGVY |
| | | PPLAVLEVEVGIGEDWLSAKE |
| 6 | TaqIT_V493L_T664I | MLLHEFGLLESPKALEEAPWPPPEGAFVGFVLSRKEPM |
| | | WADLLALAAARGGRVHRAPEPYKALRDLKEARGLLAKD |
| | | LSVLALREGLGLPPGDDPMLLAYLLDPSNTTPEGVARR |
| | | YGGEWTEEAGERAALSERLFANLWGRLEGEERLLWLYR |
| | | EVERPLSAVLAHMEATGVRLDVAYLRALSLEVAEEIAR |
| | | LEAEVFRLAGHPFNLNSRDQLERLLFDELGLPAIGKTE |
| | | KTGKRSTSAAVLEALREAHPIVEKILQYRELTKLKSTY |
| | | IDPLPDLIHPRTGRLHTRFNQTATATGRLSSSDPNLQN |
| | | IPVRTPLGQRIRRAFIAEEGWLLVALDYSQIELRVLAH |
| | | LSGDENLIRVFQEGRDIHTETASWMFGVPREAVDPLMR |
| | | RAAKIINFGVLYGMSAHRLSQELAIPYEEAQAFIERYF |
| | | QSFPKVRAWIEKTLEEGRRRGYVETLFGRRRYVPDLEA |
| | | RVKSVREAAERMAFNMPVQGTAADLMKLAMVKLFPRLE |
| | | EMGARMLLQVHDELVLEAPKERAEAVARLAKEVMEGVY |
| | | PLAVPLEVEVGIGEDWLSAKE |
| 7 | TaqIT_G824A_W70 | MLLHEFGLLESPKALEEAPWPPPEGAFVGFVLSRKEPM |
| | 6Y_E681G | WADLLALAAARGGRVHRAPEPYKALRDLKEARGLLAKD |
| | | LSVLALREGLGLPPGDDPMLLAYLLDPSNTTPEGVARR |
| | | YGGEWTEEAGERAALSERLFANLWGRLEGEERLLWLYR |
| | | EVERPLSAVLAHMEATGVRLDVAYLRALSLEVAEEIAR |
| | | LEAEVFRLAGHPFNLNSRDQLERVLFDELGLPAIGKTE |
| | | KTGKRSTSAAVLEALREAHPIVEKILQYRELTKLKSTY |
| | | IDPLPDLIHPRTGRLHTRFNQTATATGRLSSSDPNLQN |
| | | IPVRTPLGQRIRRAFIAEEGWLLVALDYSQIELRVLAH |
| | | LSGDENLIRVFQEGRDIHTETASWMFGVPREAVDPLMR |
| | | RAAKTINFGVLYGMSAHRLSQGLAIPYEEAQAFIERYF |
| | | QSFPKVRAYIEKTLEEGRRRGYVETLFGRRRYVPDLEA |
| | | RVKSVREAAERMAFNMPVQGTAADLMKLAMVKLFPRLE |
| | | EMGARMLLQVHDELVLEAPKERAEAVARLAKEVMEGVY |
| | | PLAVPLEVEVGIAEDWLSAKE |
| 8 | TaqIT_V449F_T664I | MLLHEFGLLESPKALEEAPWPPPEGAFVGFVLSRKEPM |
| | | WADLLALAAARGGRVHRAPEPYKALRDLKEARGLLAKD |
| | | LSVLALREGLGLPPGDDPMLLAYLLDPSNTTPEGVARR |
| | | YGGEWTEEAGERAALSERLFANLWGRLEGEERLLWLYR |
| | | EVERPLSAVLAHMEATGFRLDVAYLRALSLEVAEEIAR |
| | | LEAEVFRLAGHPFNLNSRDQLERVLFDELGLPAIGKTE |
| | | KTGKRSTSAAVLEALREAHPIVEKILQYRELTKLKSTY |
| | | IDPLPDLIHPRTGRLHTRFNQTATATGRLSSSDPNLQN |
| | | IPVRTPLGQRIRRAFIAEEGWLLVALDYSQIELRVLAH |
| | | LSGDENLIRVFQEGRDIHTETASWMFGVPREAVDPLMR |
| | | RAAKIINFGVLYGMSAHRLSQELAIPYEEAQAFIERYF |
| | | QSFPKVRAWIEKTLEEGRRRGYVETLFGRRRYVPDLEA |
| | | RVKSVREAAERMAFNMPVQGTAADLMKLAMVKLFPRLE |
| | | EMGARMLLQVHDELVLEAPKERAEAVARLAKEVMEGVY |
| | | PLAVPLEVEVGIGEDWLSAKE |
| 9 | TaqIT_L522I_R736K | MLLHEFGLLESPKALEEAPWPPPEGAFVGFVLSRKEPM |
| | | WADLLALAAARGGRVHRAPEPYKALRDLKEARGLLAKD |
| | | LSVLALREGLGLPPGDDPMLLAYLLDPSNTTPEGVARR |
| | | YGGEWTEEAGERAALSERLFANLWGRLEGEERLLWLYR |
| | | EVERPLSAVLAHMEATGVRLDVAYLRALSLEVAEEIAR |
| | | LEAEVFRLAGHPFNLNSRDQLERVLFDELGLPAIGKTE |
| | | KTGKRSTSAAVLEAIREAHPIVEKILQYRELTKLKSTY |
| | | IDPLPDLIHPRTGRLHTRFNQTATATGRLSSSDPNLQN |
| | | IPVRTPLGQRIRRAFIAEEGWLLVALDYSQIELRVLAH |
| | | LSGDENLIRVFQEGRDIHTETASWMFGVPREAVDPLMR |
| | | RAAKTINFGVLYGMSAHRLSQELAIPYEEAQAFIERYF |
| | | QSFPKVRAWIEKTLEEGRRRGYVETLFGRRRYVPDLEA |
| | | KVKSVREAAERMAFNMPVQGTAADLMKLAMVKLFPRLE |
| | | EMGARMLLQVHDELVLEAPKERAEAVARLAKEVMEGVY |
| | | PLAVPLEVEVGIGEDWLSAKE |

An enzyme provided herein may comprise a sequence having homology or similarity with SEQ ID NO: 1. In some instances, an enzyme provided herein comprises at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 1. In some instances, at least 10 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 1. In some instances, at least 50 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 1. In some instances, at least 100 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 1. In some instances, 20-100 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 1.

An enzyme provided herein may comprise a sequence having homology or similarity with SEQ ID NO: 2. In some instances, an enzyme provided herein comprises at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 2. In some instances, at least 10 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 2. In some instances, at least 50 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 2. In some instances, at least 100 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 2. In some instances, 20-100 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 2.

An enzyme provided herein may comprise a sequence having homology or similarity with SEQ ID NO: 3. In some instances, an enzyme provided herein comprises at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 3. In some instances, at least 10 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 3. In some instances, at least 50 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 3. In some instances, at least 100 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 3. In some instances, 20-100 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 3.

An enzyme provided herein may comprise a sequence having homology or similarity with SEQ ID NO: 4. In some instances, an enzyme provided herein comprises at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 4. In some instances, at least 10 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 4. In some instances, at least 50 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 4. In some instances, at least 100 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 4. In some instances, 20-100 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 4.

An enzyme provided herein may comprise a sequence having homology or similarity with SEQ ID NO: 5. In some instances, an enzyme provided herein comprises at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 5. In some instances, at least 10 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 5. In some instances, at least 50 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 5. In some instances, at least 100 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 5. In some instances, 20-100 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 5.

An enzyme provided herein may comprise a sequence having homology or similarity with SEQ ID NO: 6. In some instances, an enzyme provided herein comprises at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 6. In some instances, at least 10 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 6. In some instances, at least 50 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 6. In some instances, at least 100 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 6. In some instances, 20-100 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 6.

An enzyme provided herein may comprise a sequence having homology or similarity with SEQ ID NO: 7. In some instances, an enzyme provided herein comprises at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 7. In some instances, at least 10 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 7. In some instances, at least 50 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 7. In some instances, at least 100 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 7. In some instances, 20-100 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 7.

An enzyme provided herein may comprise a sequence having homology or similarity with SEQ ID NO: 8. In some instances, an enzyme provided herein comprises at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 8. In some instances, at least 10 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 8. In some instances, at least 50 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 8. In some instances, at least 100 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 8. In some instances, 20-100 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 8.

An enzyme provided herein may comprise a sequence having homology or similarity with SEQ ID NO: 9. In some instances, an enzyme provided herein comprises at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 9. In some instances, at least 10 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 9. In some instances, at least 50 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 9. In some instances, at least 100 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 9. In some instances, 20-100 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO: 9.

### Enzyme Optimization

Described herein are methods and systems of in silico library design. For example, an amino acid sequence of an enzyme or enzyme fragment may be used as input. An amino acid sequence of any enzyme may be used for input in the methods and systems described herein. A database comprising known mutations from an organism may be queried, and a library of sequences comprising combinations of these mutations may be generated. In some instances, specific mutations or combinations of mutations may be excluded from the library (e.g., known immunogenic sites, structure sites, etc.). In some instances, specific sites in the input sequence may be systematically replaced with histidine, aspartic acid, glutamic acid, or combinations thereof. In some instances, the maximum or minimum number of mutations allowed for each region of an enzyme may be specified. In some instances, mutations are described relative to the input sequence or the input sequence's corresponding germline (wild-type) sequence. For example, sequences generated by the optimization may comprise at least 1, at least 2, at least 3, at least 4, at least 5, at least 6, at least 7, at least 8, at least 9, at least 10, at least 11, at least 12, at least 13, at least 14, at least 15, at least 16, or more than 16 mutations from the input sequence. In some instances, sequences generated by the optimization comprise no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, or no more than 18 mutations from the input sequence. In some instances, sequences generated by the optimization comprise about 1, about 2, about 3, about 4, about 5, about 6, about 7, about 8, about 9, about 10, about 11, about 12, about 13, about 14, about 15, about 16, or about 18 mutations relative to the input sequence. In some instances, in silico enzyme libraries may be synthesized, assembled, and/or enriched for desired sequences.

Germline sequences corresponding to an input sequence may also be modified to generate sequences in a library. For example, sequences generated by the optimization methods described herein comprise at least 1, at least 2, at least 3, at least 4, at least 5, at least 6, at least 7, at least 8, at least 9, at least 10, at least 11, at least 12, at least 13, at least 14, at least 15, at least 16, or more than 16 mutations from the germline sequence. In some instances, sequences generated by the optimization comprise no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16or no more than 18 mutations from the germline sequence. In some instances, sequences generated by the optimization comprise about 1, about 2, about 3, about 4, about 5, about 6, about 7, about 8, about 9, about 10, about 11, about 12, about 13, about 14, about 15, about 16, or about 18 mutations relative to the germline sequence.

### Machine Learning

Data from preprocessing operations, as described herein, may be fed into one or more machine learning (ML) algorithms for identifying a library comprising one or more candidates with high affinity to a target and/or functional activity. In some embodiments, the one or more candidates comprise one or more sequences encoding for an enzyme. In some examples, the library may be a synthetic library. In some embodiments, the ML algorithms may be integrated into a computational pipeline for intelligent decision making and/or experimental validation. In some embodiments, the one or more ML algorithms may be supervised, semi-supervised, or unsupervised for training to identify anomalies. In some embodiments, the one or more ML algorithms may perform classification or clustering to identify anomalies or attacks. In some embodiments, the one or more ML algorithms may comprise classical ML algorithms for performing clustering to identify outliers. Classical ML algorithms may comprise of algorithms that learn from existing observations (i.e., known features) to predict outputs. In some cases, the classical ML algorithms for performing clustering may be K-means clustering, mean-shift clustering, density-based spatial clustering of applications with noise (DBSCAN), expectation-maximization (EM) clustering (e.g., using Gaussian mixture models (GMM)), agglomerative hierarchical clustering, or a combination thereof. In some embodiments, the one or more ML algorithms may comprise classical ML algorithms for classification. In some cases, the classical ML algorithms may comprise logistic regression, naïve Bayes, K-nearest neighbors, random forests or decision trees, gradient boosting, support vector machines (SVMs), or a combination thereof. In some embodiments, the one or more ML algorithm may employ deep learning. A deep learning algorithm may comprise of an algorithm that learns by extracting new features to predict outputs. The deep learning algorithm may comprise of layers, which may comprise a neural network.

### Expression Systems

Provided herein are libraries comprising nucleic acids encoding for enzymes, wherein the libraries have improved specificity, stability, expression, folding, or downstream activity. In some instances, libraries described herein may be used for screening and analysis.

Provided herein are libraries comprising nucleic acids encoding for enzymes, wherein the nucleic acid libraries are used for screening and analysis. In some instances, screening and analysis comprises in vitro, in vivo, or ex vivo assays. Cells for screening include primary cells taken from living subjects or cell lines. Cells may be from prokaryotes (e.g., bacteria and fungi) or eukaryotes (e.g., animals and plants). Exemplary animal cells include, without limitation, those from a mouse, rabbit, primate, and insect. In some instances, cells for screening include a cell line including, but not limited to, Chinese Hamster Ovary (CHO) cell line, human embryonic kidney (HEK) cell line, or baby hamster kidney (BHK) cell line. In some instances, nucleic acid libraries described herein may also be delivered to a multicellular organism. Exemplary multicellular organisms include, without limitation, a plant, a mouse, a rat, a rabbit, a primate (e.g., a monkey or an ape), a fish, a worm, a bird, a chicken, a camelid, a cat, a dog, a horse, a cow, a sheep, a goat, a frog, or an insect.

Nucleic acid libraries described herein may be screened for various pharmacological or pharmacokinetic properties. In some instances, the libraries are screened using in vitro assays, in vivo assays, or ex vivo assays. For example, in vitro pharmacological or pharmacokinetic properties that are screened include, but are not limited to, binding affinity, binding specificity, and binding avidity. Exemplary in vivo pharmacological or pharmacokinetic properties of libraries described herein that are screened include, but are not limited to, therapeutic efficacy, activity, preclinical toxicity properties, clinical efficacy properties, clinical toxicity properties, immunogenicity, potency, and clinical safety properties.

Provided herein are nucleic acid libraries, wherein the nucleic acid libraries may be expressed in a vector. Expression vectors for inserting nucleic acid libraries disclosed herein may comprise eukaryotic or prokaryotic expression vectors. Exemplary expression vectors include, without limitation, mammalian expression vectors: pSF-CMV-NEO-NH2-PPT-3XFLAG, pSF-CMV-NEO-COOH-3XFLAG, pSF-CMV-PURO-NH2-GST-TEV, pSF-OXB20-COOH)-TEV-FLAG (R)-6His, pCEP4 pDEST27, pSF-CMV-Ub-KrYFP, pSF-CMV-FMDV-daGFP, pEFla-mCherry-N1 Vector, pEFla-tdTomato Vector, pSF-CMV-FMDV-Hygro, pSF-CMV-PGK-Puro, pMCP-tag (m), and pSF-CMV-PURO-NH2-CMYC; bacterial expression vectors: pSF-OXB20-BetaGal,pSF-OXB20-Fluc, pSF-OXB20, and pSF-Tac; plant expression vectors: pRI 101-AN DNA and pCambia2301; and yeast expression vectors: pTYB21 and pKLAC2, and insect vectors: pAc5.1/V5-His A and pDEST8. In some instances, the vector is pcDNA3 or pcDNA3.1.

Described herein are nucleic acid libraries that are expressed in a vector to generate a construct comprising an enzyme. In some instances, a size of the construct varies. In some instances, the construct comprises at least or about 500, at least or about 600, at least or about 700, at least or about 800, at least or about 900, at least or about 1000, at least or about 1100, at least or about 1300, at least or about 1400, at least or about 1500, at least or about 1600, at least or about 1700, at least or about 1800, at least or about 2000, at least or about 2400, at least or about 2600, at least or about 2800, at least or about 3000, at least or about 3200, at least or about 3400, at least or about 3600, at least or about 3800, at least or about 4000, at least or about 4200, at least or about 4400, at least or about 4600, at least or about 4800, at least or about 5000, at least or about 6000, at least or about 7000, at least or about 8000, at least or about 9000, at least or about 10000, or more than 10000 bases. In some instances, a the construct comprises a range of about 300 to 1,000, 300 to 2,000, 300 to 3,000, 300 to 4,000, 300 to 5,000, 300 to 6,000, 300 to 7,000, 300 to 8,000, 300 to 9,000, 300 to 10,000, 1,000 to 2,000, 1,000 to 3,000, 1,000 to 4,000, 1,000 to 5,000, 1,000 to 6,000, 1,000 to 7,000, 1,000 to 8,000, 1,000 to 9,000, 1,000 to 10,000, 2,000 to 3,000, 2,000 to 4,000, 2,000 to 5,000, 2,000 to 6,000, 2,000 to 7,000, 2,000 to 8,000, 2,000 to 9,000, 2,000 to 10,000, 3,000 to 4,000, 3,000 to 5,000, 3,000 to 6,000, 3,000 to 7,000, 3,000 to 8,000, 3,000 to 9,000, 3,000 to 10,000, 4,000 to 5,000, 4,000 to 6,000, 4,000 to 7,000, 4,000 to 8,000, 4,000 to 9,000, 4,000 to 10,000, 5,000 to 6,000, 5,000 to 7,000, 5,000 to 8,000, 5,000 to 9,000, 5,000 to 10,000, 6,000 to 7,000, 6,000 to 8,000, 6,000 to 9,000, 6,000 to 10,000, 7,000 to 8,000, 7,000 to 9,000, 7,000 to 10,000, 8,000 to 9,000, 8,000 to 10,000, or 9,000 to 10,000 bases.

Provided herein are libraries comprising nucleic acids encoding for enzymes, wherein the nucleic acid libraries are expressed in a cell. In some instances, the libraries are synthesized to express a reporter gene. Exemplary reporter genes include, but are not limited to, acetohydroxyacid synthase (AHAS), alkaline phosphatase (AP), beta galactosidase (LacZ), beta glucoronidase (GUS), chloramphenicol acetyltransferase (CAT), green fluorescent protein (GFP), red fluorescent protein (RFP), yellow fluorescent protein (YFP), cyan fluorescent protein (CFP), cerulean fluorescent protein, citrine fluorescent protein, orange fluorescent protein, cherry fluorescent protein, turquoise fluorescent protein, blue fluorescent protein, horseradish peroxidase (HRP), luciferase (Luc), nopaline synthase (NOS), octopine synthase (OCS), luciferase, and derivatives thereof. Methods to determine modulation of a reporter gene are well known in the art, and include, but are not limited to, fluorometric methods (e.g. fluorescence spectroscopy, Fluorescence Activated Cell Sorting (FACS), fluorescence microscopy), and antibiotic resistance determination.

The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity.

The term “homology” or “similarity” between two proteins is determined by comparing the amino acid sequence and its conserved amino acid substitutes of one protein sequence to the second protein sequence. Similarity may be determined by procedures which are well-known in the art, for example, a BLAST program (Basic Local Alignment Search Tool at the National Center for Biological Information).

Provided herein are libraries comprising nucleic acids encoding for enzymes (e.g., polymerases). Enzymes described herein allow for improved stability for a range of active site encoding sequences. In some instances, the active site encoding sequences are determined by interactions between the substrate and the catalytically active site of an enzyme.

Sequences of active sites based on surface interactions between a ligand/substrate and an enzyme described herein are analyzed using various methods. For example, multispecies computational analysis is performed. In some instances, a structure analysis is performed. In some instances, a sequence analysis is performed. Sequence analysis can be performed using a database known in the art. Non-limiting examples of databases include, but are not limited to, NCBI BLAST (blast.ncbi.nlm.nih.gov/Blast.cgi), UCSC Genome Browser (genome.ucsc.edu/), UniProt (www.uniprot.org/), and IUPHAR/BPS Guide to PHARMACOLOGY (guidetopharmacology.org/).

Described herein are active sites designed based on sequence analysis among various organisms. For example, sequence analysis is performed to identify homologous sequences in different organisms. Exemplary organisms include, but are not limited to, mouse, rat, equine, sheep, cow, primate (e.g., chimpanzee, baboon, gorilla, orangutan, monkey), dog, cat, pig, donkey, rabbit, camelid, fish, fly, or human. In some instances, homologous sequences are identified in the same organism, across individuals.

Following identification of active sites, libraries comprising nucleic acids encoding for the active sites may be generated. In some instances, libraries of active sites comprise sequences of active sites designed based on conformational ligand/substrate interactions. Libraries of active sites may be translated to generate protein libraries. In some instances, libraries of active sites are translated to generate peptide libraries, immunoglobulin libraries, derivatives thereof, or combinations thereof. In some instances, libraries of active sites are translated to generate protein libraries that are further modified to generate peptidomimetic libraries. In some instances, libraries of active sites are translated to generate protein libraries that are used to generate small molecules.

Methods described herein provide for synthesis of libraries of active sites comprising nucleic acids each encoding for a predetermined variant of at least one predetermined reference nucleic acid sequence. In some cases, the predetermined reference sequence is a nucleic acid sequence encoding for a protein, and the variant library comprises sequences encoding for variation of at least a single codon such that a plurality of different variants of a single residue in the subsequent protein encoded by the synthesized nucleic acid are generated by standard translation processes. In some instances, the libraries of active sites comprise varied nucleic acids collectively encoding variations at multiple positions. In some instances, the variant library comprises sequences encoding for variation of at least a single codon in an active site. In some instances, the variant library comprises sequences encoding for variation of multiple codons in an active site. An exemplary number of codons for variation include, but are not limited to, at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons.

Methods described herein provide for synthesis of libraries comprising nucleic acids encoding for the active sites, wherein the libraries comprise sequences encoding for variation of length of the active sites. In some instances, the library comprises sequences encoding for variation of length of at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons less as compared to a predetermined reference sequence. In some instances, the library comprises sequences encoding for variation of length of at least or about 1, 5, 10, 15, 20, 25, 30, 35, 40, 45, 50, 55, 60, 65, 70, 75, 80, 85, 90, 95, 100, 125, 150, 175, 200, 225, 250, 275, 300, or more than 300 codons more as compared to a predetermined reference sequence.

Following identification of active sites, enzymes may be designed and synthesized to comprise the active sites. Enzymes comprising active sites may be designed based on binding, specificity, stability, expression, folding, or downstream activity.

Methods described herein provide for synthesis of a library of nucleic acids each encoding for a predetermined variant of at least one predetermined reference nucleic acid sequence. In some cases, the predetermined reference sequence is a nucleic acid sequence encoding for a protein, and the variant library comprises sequences encoding for variation of at least a single codon such that a plurality of different variants of a single residue in the subsequent protein encoded by the synthesized nucleic acid are generated by standard translation processes. In some instances, the library comprises varied nucleic acids collectively encoding variations at multiple positions. In some instances, the variant library comprises sequences encoding for variation of at least a single codon in an active site. For example, at least one single codon of the enzyme is varied. An exemplary number of codons for variation include, but are not limited to, at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons.

Methods described herein provide for synthesis of a library of nucleic acids each encoding for a predetermined variant of at least one predetermined reference nucleic acid sequence, wherein the library comprises sequences encoding for variation of length of a domain in the enzyme. In some instances, the library comprises sequences encoding for variation of length of at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons less as compared to a predetermined reference sequence. In some instances, the library comprises sequences encoding for variation of length of at least or about 1, 5, 10, 15, 20, 25, 30, 35, 40, 45, 50, 55, 60, 65, 70, 75, 80, 85, 90, 95, 100, 125, 150, 175, 200, 225, 250, 275, 300, or more than 300 codons more as compared to a predetermined reference sequence.

Following synthesis of enzyme libraries for screening and analysis. For example, libraries are assayed for library displayability, screening, and/or panning. In some instances, displayability is assayed using a selectable tag. Exemplary tags include, but are not limited to, a radioactive label, a fluorescent label, an enzyme, a chemiluminescent tag, a colorimetric tag, an affinity tag or other labels or tags that are known in the art. In some instances, the tag is histidine, polyhistidine, myc, hemagglutinin (HA), or FLAG. In some instances, libraries are assayed by sequencing using various methods including, but not limited to, single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. In instances, libraries are assayed for A-tailing activity or stability.

### Variant Libraries

### Codon Variation

Variant nucleic acid libraries described herein may comprise a plurality of nucleic acids, wherein each nucleic acid encodes for a variant codon sequence compared to a reference nucleic acid sequence. In some instances, each nucleic acid of a first nucleic acid population contains a variant at a single variant site. In some instances, the first nucleic acid population contains a plurality of variants at a single variant site such that the first nucleic acid population contains more than one variant at the same variant site. The first nucleic acid population may comprise nucleic acids collectively encoding multiple codon variants at the same variant site. The first nucleic acid population may comprise nucleic acids collectively encoding up to 19 or more codons at the same position. The first nucleic acid population may comprise nucleic acids collectively encoding up to 60 variant triplets at the same position, or the first nucleic acid population may comprise nucleic acids collectively encoding up to 61 different triplets of codons at the same position. Each variant may encode for a codon that results in a different amino acid during translation. Table 2 provides a listing of each codon possible (and the representative amino acid) for a variant site.

TABLE 2
List of Codons and Amino Acid Residues

| | 1-letter | 3-letter | | | | | | |
|---|---|---|---|---|---|---|---|---|
| Amino Acids | code | code | Codons | | | | | |
| Alanine | A | Ala | GCA | GCC | GCG | GCT | | |
| Cysteine | C | Cys | TGC | TGT | | | | |
| Aspartic acid | D | Asp | GAC | GAT | | | | |
| Glutamic acid | E | Glu | GAA | GAG | | | | |
| Phenylalanine | F | Phe | TTC | TTT | | | | |
| Glycine | G | Gly | GGA | GGC | GGG | GGT | | |
| Histidine | H | His | CAC | CAT | | | | |
| Isoleucine | I | Iso | ATA | ATC | ATT | | | |
| Lysine | K | Lys | AAA | AAG | | | | |
| Leucine | L | Leu | TTA | TTG | CTA | CTC | CTG | CTT |
| Methionine | M | Met | ATG | | | | | |
| Asparagine | N | Asn | AAC | AAT | | | | |
| Proline | P | Pro | CCA | CCC | CCG | CCT | | |
| Glutamine | Q | Gln | CAA | CAG | | | | |
| Arginine | R | Arg | AGA | AGG | CGA | CGC | CGG | CGT |
| Serine | S | Ser | AGC | AGT | TCA | TCC | TCG | TCT |

| | | | | | | |
|---|---|---|---|---|---|---|
| Threonine | T | Thr | ACA | ACC | ACG | ACT |
| Valine | V | Val | GTA | GTC | GTG | GTT |

| Tryptophan | W | Trp | TGG | |
|---|---|---|---|---|
| Tyrosine | Y | Tyr | TAC | TAT |

A nucleic acid population may comprise varied nucleic acids collectively encoding up to 20 codon variations at multiple positions. In such cases, each nucleic acid in the population comprises variation for codons at more than one position in the same nucleic acid. In some instances, each nucleic acid in the population comprises variation for codons at 1, 2, 3, 4, 5, 6, 7, 8, 9, 10, 11, 12, 13, 14, 15, 16, 17, 18, 19, 20 or more codons in a single nucleic acid. In some instances, each variant long nucleic acid comprises variation for codons at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more codons in a single long nucleic acid. In some instances, the variant nucleic acid population comprises variation for codons at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more codons in a single nucleic acid. In some instances, the variant nucleic acid population comprises variation for codons in at least about 10, 20, 30, 40, 50, 60, 70, 80, 90, 100 or more codons in a single long nucleic acid.

### Highly Parallel Nucleic Acid Synthesis

Provided herein is a platform approach utilizing miniaturization, parallelization, and vertical integration of the end-to-end process from polynucleotide synthesis to gene assembly within nanowells on silicon to create a revolutionary synthesis platform. Devices described herein provide, with the same footprint as a 96-well plate, a silicon synthesis platform is capable of increasing throughput by a factor of up to 1,000 or more compared to traditional synthesis methods, with production of up to approximately 1,000,000 or more polynucleotides, or 10,000 or more genes in a single highly-parallelized run.

With the advent of next-generation sequencing, high resolution genomic data has become an important factor for studies that delve into the biological roles of various genes in both normal biology and disease pathogenesis. At the core of this research is the central dogma of molecular biology and the concept of “residue-by-residue transfer of sequential information.” Genomic information encoded in the DNA is transcribed into a message that is then translated into the protein that is the active product within a given biological pathway.

Another exciting area of study is on the discovery, development and manufacturing of therapeutic molecules focused on a highly-specific cellular target. High diversity DNA sequence libraries are at the core of development pipelines for targeted therapeutics. Gene mutants are used to express proteins in a design, build, and test protein engineering cycle that ideally culminates in an optimized gene for high expression of a protein with high affinity for its therapeutic target. As an example, consider the binding pocket of a receptor. The ability to test all sequence permutations of all residues within the binding pocket simultaneously will allow for a thorough exploration, increasing chances of success. Saturation mutagenesis, in which a researcher attempts to generate all possible mutations at a specific site within the receptor, represents one approach to this development challenge. Though costly and time and labor-intensive, it enables each variant to be introduced into each position. In contrast, combinatorial mutagenesis, where a few selected positions or short stretch of DNA may be modified extensively, generates an incomplete repertoire of variants with biased representation.

To accelerate the drug development pipeline, a library with the desired variants available at the intended frequency in the right position available for testing—in other words, a precision library, enables reduced costs as well as turnaround time for screening. Provided herein are methods for synthesizing nucleic acid synthetic variant libraries which provide for precise introduction of each intended variant at the desired frequency. To the end user, this translates to the ability to not only thoroughly sample sequence space but also be able to query these hypotheses in an efficient manner, reducing cost and screening time. Genome-wide editing can elucidate important pathways, libraries where each variant and sequence permutation can be tested for optimal functionality, and thousands of genes can be used to reconstruct entire pathways and genomes to re-engineer biological systems for drug discovery.

In a first example, an enzyme itself can be optimized using methods described herein. For example, to improve a specified function of an enzyme, a variant polynucleotide library encoding for a portion of the enzyme is designed and synthesized. A variant nucleic acid library for the enzyme can then be generated by processes described herein (e.g., PCR mutagenesis followed by insertion into a vector). The enzyme is then expressed in a production cell line and screened for enhanced activity. Example screens include examining modulation in binding affinity to a substrate, stability (e.g., heat, salt), or function (e.g., substrate scope, speed).

Nucleic acid libraries synthesized by methods described herein may be expressed in various cells associated with a disease state. Cells associated with a disease state include cell lines, tissue samples, primary cells from a subject, cultured cells expanded from a subject, or cells in a model system. Exemplary model systems include, without limitation, plant and animal models of a disease state.

To identify a variant molecule associated with prevention, reduction or treatment of a disease state, a variant nucleic acid library described herein is expressed in a cell associated with a disease state, or one in which a cell a disease state can be induced. In some instances, an agent is used to induce a disease state in cells. Exemplary tools for disease state induction include, without limitation, a Cre/Lox recombination system, LPS inflammation induction, and streptozotocin to induce hypoglycemia. The cells associated with a disease state may be cells from a model system or cultured cells, as well as cells from a subject having a particular disease condition. Exemplary disease conditions include a bacterial, fungal, viral, autoimmune, or proliferative disorder (e.g., cancer). In some instances, the variant nucleic acid library is expressed in the model system, cell line, or primary cells derived from a subject, and screened for changes in at least one cellular activity. Exemplary cellular activities include, without limitation, proliferation, cycle progression, cell death, adhesion, migration, reproduction, cell signaling, energy production, oxygen utilization, metabolic activity, and aging, response to free radical damage, or any combination thereof.

In some instances, methods described herein provide for generation of a library of nucleic acids comprising variant nucleic acids differing at a plurality of codon sites. In some instances, a nucleic acid may have 1 site, 2 sites, 3 sites, 4 sites, 5 sites, 6 sites, 7 sites, 8 sites, 9 sites, 10 sites, 11 sites, 12 sites, 13 sites, 14 sites, 15 sites, 16 sites, 17 sites 18 sites, 19 sites, 20 sites, 30 sites, 40 sites, 50 sites, or more of variant codon sites. In some instances, the one or more sites of variant codon sites may be adjacent. In some instances, the one or more sites of variant codon sites may not be adjacent and separated by 1, 2, 3, 4, 5, 6, 7, 8, 9, 10, or more codons. In some instances, a nucleic acid may comprise multiple sites of variant codon sites, wherein all the variant codon sites are adjacent to one another, forming a stretch of variant codon sites. In some instances, a nucleic acid may comprise multiple sites of variant codon sites, wherein none the variant codon sites are adjacent to one another. In some instances, a nucleic acid may comprise multiple sites of variant codon sites, wherein some the variant codon sites are adjacent to one another, forming a stretch of variant codon sites, and some of the variant codon sites are not adjacent to one another.

### Sequencing

Enzymes provided herein may be used for a variety of downstream applications. In some instances, enzymes comprise polymerases. In one some instances, a sample is obtained from one or more sources, and the population of sample polynucleotides is isolated. Samples are obtained (by way of non-limiting example) from biological sources such as saliva, blood, tissue, skin, or completely synthetic sources. In some instances, samples comprise circulating tumor DNA (ctDNA), cell-free DNA (cfDNA), or other nucleic acid sample. The plurality of polynucleotides obtained from the sample are fragmented, end-repaired, and adenylated to form a double stranded sample nucleic acid fragment. In some instances, end repair is accomplished by treatment with one or more enzymes, such as a T4 DNA polymerase or variant thereof (including Taq variants described herein), klenow enzyme, and T4 polynucleotide kinase in an appropriate buffer. A nucleotide overhang to facilitate ligation to adapters is added, in some instances with 3′ to 5′ exo minus klenow fragment and dATP. A nucleotide overhang to facilitate ligation to adapters is added, in some instances with a variant polymerase described herein and dATP.

Adapters (such as universal adapters) may be ligated to both ends of the sample polynucleotide fragments with a ligase, such as T4 ligase described herein, to produce a library of adapter-tagged polynucleotide strands, and the adapter-tagged polynucleotide library is amplified with primers, such as universal primers. In some instances, the adapters are Y-shaped adapters comprising one or more primer binding sites, one or more grafting regions, and one or more index (or barcode) regions. In some instances, the one or more index region is present on each strand of the adapter. In some instances, grafting regions are complementary to a flow cell surface, and facilitate next generation sequencing of sample libraries. In some instances, Y-shaped adapters comprise partially complementary sequences. In some instances, Y-shaped adapters comprise a single thymidine overhang which hybridizes to the overhanging adenine of the double stranded adapter-tagged polynucleotide strands. Y-shaped adapters may comprise modified nucleic acids, that are resistant to cleavage. For example, a phosphorothioate backbone is used to attach an overhanging thymidine to the 3′ end of the adapters. If universal primers are used, amplification of the library is performed to add barcoded primers to the adapters.

A plurality of nucleic acids (i.e. genomic sequence) may be obtained from a sample, and fragmented, optionally end-repaired, and adenylated. Adapters are ligated to both ends of the polynucleotide fragments to produce a library of adapter-tagged polynucleotide strands, and the adapter-tagged polynucleotide library is amplified. The adapter-tagged polynucleotide library is then denatured at high temperature, preferably 96° C., in the presence of adapter blockers. A polynucleotide targeting library (probe library) is denatured in a hybridization solution at high temperature, preferably about 90° C. to 99° C., and combined with the denatured, tagged polynucleotide library in hybridization solution for about 10 hours to 24 hours at about 45° C. to 80° C. Binding buffer is then added to the hybridized tagged polynucleotide probes, and a solid support comprising a capture moiety are used to selectively bind the hybridized adapter-tagged polynucleotide-probes. The solid support is washed one or more times with buffer, preferably about 2 and 5 times to remove unbound polynucleotides before an elution buffer is added to release the enriched, adapter-tagged polynucleotide fragments from the solid support. The enriched library of adapter-tagged polynucleotide fragments is amplified and then the library is sequenced. Alternative variables such as incubation times, temperatures, reaction volumes/concentrations, number of washes, or other variables consistent with the specification are also employed in the method.

In any of the instances, the detection or quantification analysis of the oligonucleotides can be accomplished by sequencing. The subunits or entire synthesized oligonucleotides can be detected via full sequencing of all oligonucleotides by any suitable methods known in the art, e.g., Illumina sequencing by synthesis, PacBio SMRT sequencing (waveguide), Oxford Nanopore (nanopore sequencing) or BGI/MGI nanoball sequencing, including the sequencing methods described herein.

Sequencing can be accomplished through classic Sanger sequencing methods which are well known in the art. Sequencing can also be accomplished using high-throughput systems some of which allow detection of a sequenced nucleotide immediately after or upon its incorporation into a growing strand, i.e., detection of sequence in red time or substantially real time. In some cases, high throughput sequencing generates at least 1,000, at least 5,000, at least 10,000, at least 20,000, at least 30,000, at least 40,000, at least 50,000, at least 100,000 or at least 500,000 sequence reads per hour; with each read being at least 50, at least 60, at least 70, at least 80, at least 90, at least 100, at least 120 or at least 150 bases per read.

In some instances, high-throughput sequencing involves the use of technology available by Illumina's Genome Analyzer IIX, MiSeq personal sequencer, or HiSeq systems, such as those using HiSeq 2500, HiSeq 1500, HiSeq 2000, HiSeq 1000, iSeq 100, Mini Seq, MiSeq, NextSeq 550, NextSeq 2000, NextSeq 550, or NovaSeq 6000. These machines use reversible terminator-based sequencing by synthesis chemistry. These machines can generate 6000 Gb or more reads in 13-44 hours. Smaller systems may be utilized for runs within 3, 2, 1 days or less time. Short synthesis cycles may be used to minimize the time it takes to obtain sequencing results.

In some instances, high-throughput sequencing involves the use of technology available by ABI Solid System. This genetic analysis platform that enables massively parallel sequencing of clonally-amplified DNA fragments linked to beads. The sequencing methodology is based on sequential ligation with dye-labeled oligonucleotides.

The next generation sequencing can comprise ion semiconductor sequencing (e.g., using technology from Life Technologies (Ion Torrent)). Ion semiconductor sequencing can take advantage of the fact that when a nucleotide is incorporated into a strand of DNA, an ion can be released. To perform ion semiconductor sequencing, a high density array of micromachined wells can be formed. Each well can hold a single DNA template. Beneath the well can be an ion sensitive layer, and beneath the ion sensitive layer can be an ion sensor. When a nucleotide is added to a DNA, H+ can be released, which can be measured as a change in pH. The H+ ion can be converted to voltage and recorded by the semiconductor sensor. An array chip can be sequentially flooded with one nucleotide after another. No scanning, light, or cameras can be required. In some cases, an IONPROTON™ Sequencer is used to sequence nucleic acid. In some cases, an IONPGM™ Sequencer is used. The Ion Torrent Personal Genome Machine (PGM) can do 10 million reads in two hours.

In some instances, high-throughput sequencing involves the use of technology available by Helicos BioSciences Corporation (Cambridge, Mass.) such as the Single Molecule Sequencing by Synthesis (SMSS) method. SMSS is unique because it allows for sequencing the entire human genome in up to 24 hours. Finally, SMSS is powerful because, like the MW technology, it does not require a pre amplification step prior to hybridization. In fact, SMSS does not require any amplification. SMSS is described in part in US Publication application Nos. 2006002471 I; 20060024678; 20060012793; 20060012784; and 20050100932.

In some instances, high-throughput sequencing involves the use of technology available by 454 Lifesciences, Inc. (Branford, Conn.) such as the Pico Titer Plate device which includes a fiber optic plate that transmits chemiluminescent signal generated by the sequencing reaction to be recorded by a CCD camera in the instrument. This use of fiber optics allows for the detection of a minimum of 20 million base pairs in 4.5 hours.

Methods for using bead amplification followed by fiber optics detection are described in Marguiles, et al., “Genome sequencing in microfabricated high-density picolitre reactors”, Nature, 2005, vol. 437, pages 376-380; and well as in U.S. Publication Nos. 2002/0012930; 2003/0058629, 2003/0100102, 2003/0148344, 2004/0248161, 2005/0079510, 2005/0124022, and 2006/0078909.

In some instances, high-throughput sequencing is performed using Clonal Single Molecule Array (Solexa, Inc.) or sequencing-by-synthesis (SBS) utilizing reversible terminator chemistry. These technologies are described in part in U.S. Pat. Nos. 6,969,488; 6,897,023; 6,833,246; 6,787,308; and U.S. Publication Nos. 2004/0106130, 2003/0064398, 2003/0022207; and Constans, “Beyond Sanger: toward the $1,000 genome: new technologies promise faster and cheaper whole-genome sequencing”, The Scientist, 2003, vol. 17, no. 13, pages 36+. High-throughput sequencing of oligonucleotides can be achieved using any suitable sequencing method known in the art, such as those commercialized by Pacific Biosciences, Complete Genomics, Genia Technologies, Halcyon Molecular, Oxford Nanopore Technologies and the like. Other high-throughput sequencing systems include those disclosed in Venter, et al., Science, 2001; Adams, et al., Science, 2000; and Levene, et al., Science, 2003, vol. 299, pages 682-686; as well as U.S. Publication Nos. 2003/0044781 and 2006/0078937. Overall, such systems involve sequencing a target oligonucleotide molecule having a plurality of bases by the temporal addition of bases via a polymerization reaction that is measured on a molecule of oligonucleotide, i.e., the activity of a nucleic acid polymerizing enzyme on the template oligonucleotide molecule to be sequenced is followed in real time. Sequence can then be deduced by identifying which base is being incorporated into the growing complementary strand of the target oligonucleotide by the catalytic activity of the nucleic acid polymerizing enzyme at each step in the sequence of base additions. A polymerase on the target oligonucleotide molecule complex is provided in a position suitable to move along the target oligonucleotide molecule and extend the oligonucleotide primer at an active site. A plurality of labeled types of nucleotide analogs are provided proximate to the active site, with each distinguishably type of nucleotide analog being complementary to a different nucleotide in the target oligonucleotide sequence. The growing oligonucleotide strand is extended by using the polymerase to add a nucleotide analog to the oligonucleotide strand at the active site, where the nucleotide analog being added is complementary to the nucleotide of the target oligonucleotide at the active site. The nucleotide analog added to the oligonucleotide primer as a result of the polymerizing step is identified. The steps of providing labeled nucleotide analogs, polymerizing the growing oligonucleotide strand, and identifying the added nucleotide analog are repeated so that the oligonucleotide strand is further extended and the sequence of the target oligonucleotide is determined.

The next-generation sequencing technique can comprise real-time (SMRT™) technology by Pacific Biosciences. In SMRT, each of four DNA bases can be attached to one of four different fluorescent dyes. These dyes can be phospho-linked. A single DNA polymerase can be immobilized with a single molecule of template single-stranded DNA at the bottom of a zero-mode waveguide (ZMW). A ZMW can be a confinement structure which enables observation of incorporation of a single nucleotide by DNA polymerase against the background of fluorescent nucleotides that can rapidly diffuse in an out of the ZMW (in microseconds). It can take several milliseconds to incorporate a nucleotide into a growing strand. During this time, the fluorescent label can be excited and produce a fluorescent signal, and the fluorescent tag can be cleaved off. The ZMW can be illuminated from below. Attenuated light from an excitation beam can penetrate the lower 20-30 nm of each ZMW. A microscope with a detection limit of 20 zepto liters (10″ liters) can be created. The tiny detection volume can provide 1000-fold improvement in the reduction of background noise. Detection of the corresponding fluorescence of the dye can indicate which base was incorporated. The process can be repeated.

In some cases, the next-generation sequencing is nanopore sequencing. See, e.g., Soni, et al., Clin Chem., 2007, vol. 53, pages 1996-2001. A nanopore can be a small hole, of the order of about one nanometer in diameter. Immersion of a nanopore in a conducting fluid and application of a potential across it can result in a slight electrical current due to conduction of ions through the nanopore. The amount of current which flows can be sensitive to the size of the nanopore. As a DNA molecule passes through a nanopore, each nucleotide on the DNA molecule can obstruct the nanopore to a different degree. Thus, the change in the current passing through the nanopore as the DNA molecule passes through the nanopore can represent a reading of the DNA sequence. The nanopore sequencing technology can be from Oxford Nanopore Technologies; e.g., a GridION system. A single nanopore can be inserted in a polymer membrane across the top of a microwell. Each microwell can have an electrode for individual sensing. The microwells can be fabricated into an array chip, with 100,000 or more microwells (e.g., more than 200,000, 300,000, 400,000, 500,000, 600,000, 700,000, 800,000, 900,000, or 1,000,000) per chip. An instrument (or node) can be used to analyze the chip. Data can be analyzed in real-time. One or more instruments can be operated at a time. The nanopore can be a protein nanopore, e.g., the protein alpha-hemolysin, a heptameric protein pore. The nanopore can be a solid-state nanopore made, e.g., a nanometer sized hole formed in a synthetic membrane (e.g., SiNₓ, or SiO₂). The nanopore can be a hybrid pore (e.g., an integration of a protein pore into a solid-state membrane). The nanopore can be a nanopore with an integrated sensors (e.g., tunneling electrode detectors, capacitive detectors, or graphene based nano-gap or edge state detectors (see, e.g., Garaj, et al., Nature, 2010, vol. 467, pages 190-193). A nanopore can be functionalized for analyzing a specific type of molecule (e.g., DNA, RNA, or protein). Nanopore sequencing can comprise “strand sequencing” in which intact DNA polymers can be passed through a protein nanopore with sequencing in real time as the DNA translocates the pore. An enzyme can separate strands of a double stranded DNA and feed a strand through a nanopore. The DNA can have a hairpin at one end, and the system can read both strands. In some cases, nanopore sequencing is “exonuclease sequencing” in which individual nucleotides can be cleaved from a DNA strand by a processive exonuclease, and the nucleotides can be passed through a protein nanopore. The nucleotides can transiently bind to a molecule in the pore (e.g., cyclodextran). A characteristic disruption in current can be used to identify bases.

Nanopore sequencing technology from GENIA can be used. An engineered protein pore can be embedded in a lipid bilayer membrane. “Active Control” technology can be used to enable efficient nanopore-membrane assembly and control of DNA movement through the channel. In some cases, the nanopore sequencing technology is from NABsys. Genomic DNA can be fragmented into strands of average length of about 100 kb. The 100 kb fragments can be made single stranded and subsequently hybridized with a 6-mer probe. The genomic fragments with probes can be driven through a nanopore, which can create a current-versus-time tracing. The current tracing can provide the positions of the probes on each genomic fragment. The genomic fragments can be lined up to create a probe map for the genome. The process can be done in parallel for a library of probes. A genome-length probe map for each probe can be generated. Errors can be fixed with a process termed “moving window Sequencing By Hybridization (mwSBH).” In some cases, the nanopore sequencing technology is from IBM/Roche. An electron beam can be used to make a nanopore sized opening in a microchip. An electrical field can be used to pull or thread DNA through the nanopore. A DNA transistor device in the nanopore can comprise alternating nanometer sized layers of metal and dielectric. Discrete charges in the DNA backbone can get trapped by electrical fields inside the DNA nanopore. Turning off and on gate voltages can allow the DNA sequence to be read.

The next generation sequencing can comprise DNA nanoball sequencing as performed, e.g., by Complete Genomics. See, e.g., Drmanac, et al., Science, 2010, vol. 327, no. 5961, pages 78-81. DNA can be isolated, fragmented, and size selected. For example, DNA can be fragmented (e.g., by sonication) to a mean length of about 500 bp. Adaptors (Adl) can be attached to the ends of the fragments. The adaptors can be used to hybridize to anchors for sequencing reactions. DNA with adaptors bound to each end can be PCR amplified. The adaptor sequences can be modified so that complementary single strand ends bind to each other forming circular DNA. The DNA can be methylated to protect it from cleavage by a type IIS restriction enzyme used in a subsequent step. An adaptor (e.g., the right adaptor) can have a restriction recognition site, and the restriction recognition site can remain non-methylated. The non-methylated restriction recognition site in the adaptor can be recognized by a restriction enzyme (e.g., Acul), and the DNA can be cleaved by Acul 13 bp to the right of the right adaptor to form linear double stranded DNA. A second round of right and left adaptors (Ad2) can be ligated onto either end of the linear DNA, and all DNA with both adapters bound can be PCR amplified (e.g., by PCR). Ad2 sequences can be modified to allow them to bind each other and form circular DNA. The DNA can be methylated, but a restriction enzyme recognition site can remain non-methylated on the left Adl adapter. A restriction enzyme (e.g., Acul) can be applied, and the DNA can be cleaved 13 bp to the left of the Adl to form a linear DNA fragment. A third round of right and left adaptor (Ad3) can be ligated to the right and left flank of the linear DNA, and the resulting fragment can be PCR amplified. The adaptors can be modified so that they can bind to each other and form circular DNA. A type III restriction enzyme (e.g., EcoP15) can be added; EcoP15 can cleave the DNA 26 bp to the left of Ad3 and 26 bp to the right of Ad2. This cleavage can remove a large segment of DNA and linearize the DNA once again. A fourth round of right and left adaptors (Ad4) can be ligated to the DNA, the DNA can be amplified (e.g., by PCR), and modified so that they bind each other and form the completed circular DNA template.

Rolling circle replication (e.g., using Phi 29 DNA polymerase) can be used to amplify small fragments of DNA. The four adaptor sequences can contain palindromic sequences that can hybridize and a single strand can fold onto itself to form a DNA nanoball (DNB™) which can be approximately 200-300 nanometers in diameter on average. A DNA nanoball can be attached (e.g., by adsorption) to a microarray (sequencing flowcell). The flow cell can be a silicon wafer coated with silicon dioxide, titanium and hexamethyldisilazane (HMDS) and a photoresist material. Sequencing can be performed by unchained sequencing by ligating fluorescent probes to the DNA. The color of the fluorescence of an interrogated position can be visualized by a high resolution camera. The identity of nucleotide sequences between adaptor sequences can be determined.

Provided herein are methods for preparing a nucleic acid library comprising one or more steps of providing one or more sample nucleic acids; end repair of sample nucleic acids; A-tailing of sample nucleic acids using a variant polymerase described herein, contacting the one or more sample nucleic acids with a plurality of adapters and a ligase to form a nucleic acid sequencing library comprising adapter-ligated nucleic acids; and sequencing the nucleic acid library. In some instances, the sample nucleic acids comprise genomic fragments.

In some instances, the genomic fragments are obtained from cleavage of a genome. In some instances, the genomic fragments are obtained from amplification of a genome. In some instances the sample nucleic acids comprise cDNAs. In some instances the sample nucleic acids comprise cfDNAs. In some instances the method further comprises one or more steps to prepare nucleic acid library, such as end-repair, a-tailing, and amplification. In some instances the method further comprises enriching the nucleic acid library prior to sequencing.

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### Kits

Compositions and methods provided herein may be present in a kit. In some instances a kit for nucleic library preparation comprises (a) a ligase; (b) a variant polymerase described herein; and (c) at least one adapter. In some instances, a kit comprises packaging for holding the kit components. In some instances, a kit comprises instructions for using the kit components. In some instances, a kit comprises adapters, buffers, additional enzymes, polymerases, dNTPs, or other components for use with sequencing library preparation.

### Examples

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1: Taq Polymerase High Throughput Assay

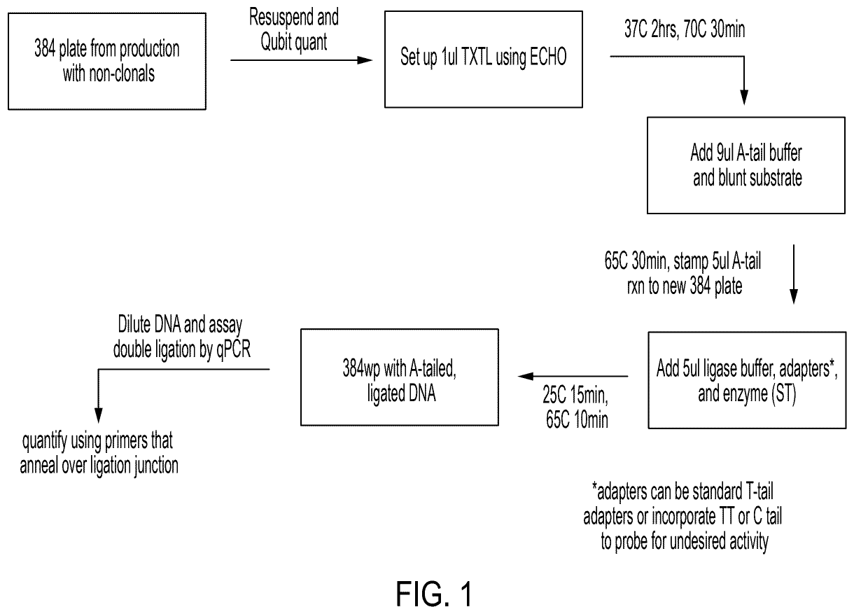

The general workflow is shown in FIG. 1. For this 384-well plate A-tailing enzyme screening protocol, non-clonal fragments were obtained from Twist Bioscience Corporation. These fragments were designed to contain T7 promoter and terminator flanking the enzyme variant sequence. This DNA came lyophilized and was resuspended in water. The DNA concentration in each well was assayed with BR dsDNA Qubit (Thermofisher). An ECHO liquid transfer instrument was used to set up small-scale, 1 μL, transcription-coupled translation (TxTl) reactions with a normalized mass of DNA template at 37° C. for 2 hours that are used to produce the enzyme variants, one unique variant in each well. After TxTI, heat treatment at 70° C. for 30 minutes, of the protein mixture was used to inactivate the TxTL proteins and leave just the TaqIT variant active (TaqIT lacks the first 280 amino acids of native Taq polymerase). A-tailing reaction was carried out with A-tailing Reaction Buffer, dNTPs, enzyme produced from TxTl and 5 ng of a blunt 230 bp DNA substrate generated by restriction enzyme digestion with MlyI. This blunt substrate is a mixture of 4 sequences that all have identical sequences except for the terminal base on either side which is an equimolar mixture of all 4 bases. The A-tailing reaction was incubated at 65C for 30 min to allow the enzyme variants to make untemplated additions to the blunt substrate. The reaction was then split in half to evaluate distinct base additions separately. To look at the desired activity of the enzyme variant standard T-tailed adapters were used to ligate to the A-tailed substrate. TT or C-tailed adapters were also used to quantify AA or G addition by the enzyme variant. Double ligation products were evaluated by qPCR after dilution of the reaction 1:300. The qPCR primers used to measure ligation anneal across the ligation junction to ensure proper ligation. In addition a separate primer pair was utilized to measure chimeric molecule ligation, an undesired outcome for this experiment. Based on the qPCR data with the respective screens, Ct values are compiled and variant hits are identified that are brought into the next round of design or which are purified for validation.

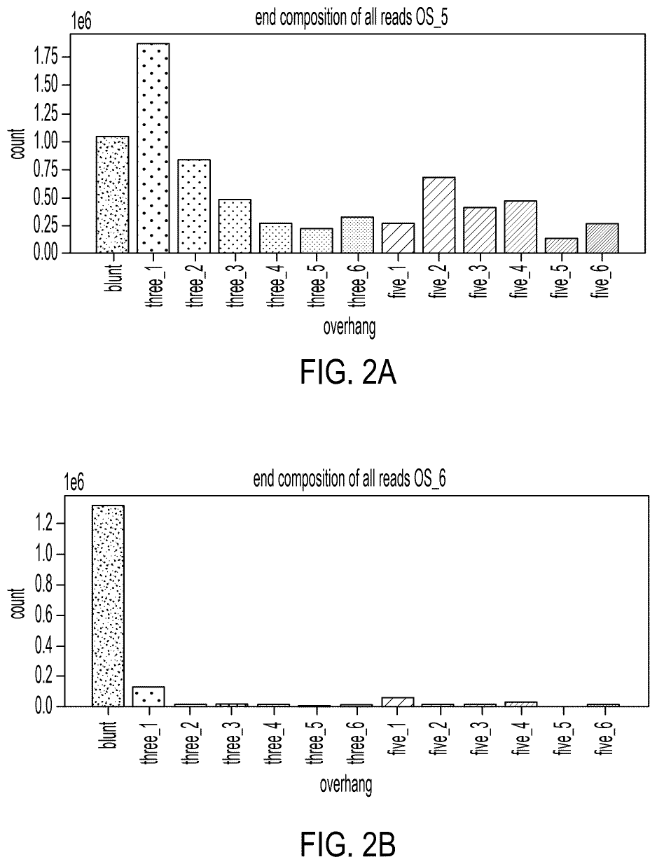

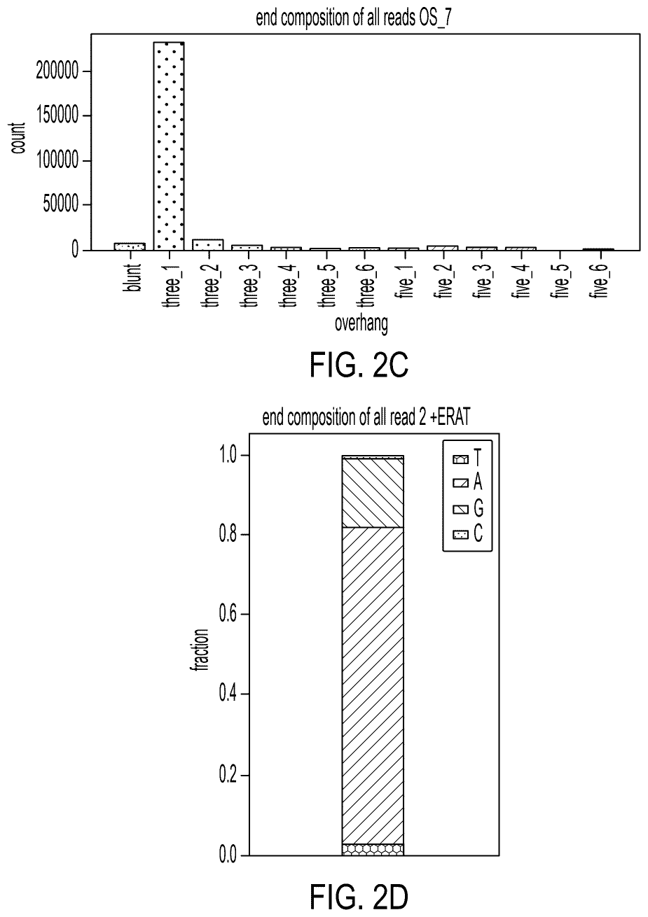

Using purified WT TaqIT the overhang and base composition of A-tailed products were evaluated. In this assay a pool of adapters with distinct overhangs and base compositions were ligated to substrates with unknown ends. Each adapter had a barcode that allows the end type of the DNA substrate (e.g. blunt end or 3-bp 5′ overhang) to be decoded following sequencing. End compositions of untreated cfDNA (FIG. 2A), cfDNA after end repair (FIG. 2B), and cfDNA after end repair and A-tailing with WT TaqIT (FIG. 2C). End repair successfully blunts the molecules, and A-tailing adds a single A to a majority of ends. In this experiment evaluating the base composition of the 3′ 1 bp overhang added by WT TaqIT, almost 20% have a base other than A added (FIG. 2D). These bases would not be available for ligation to the standard T-adapter (having a one base T overhang).

### Example 2: Taq Polymerase Optimization

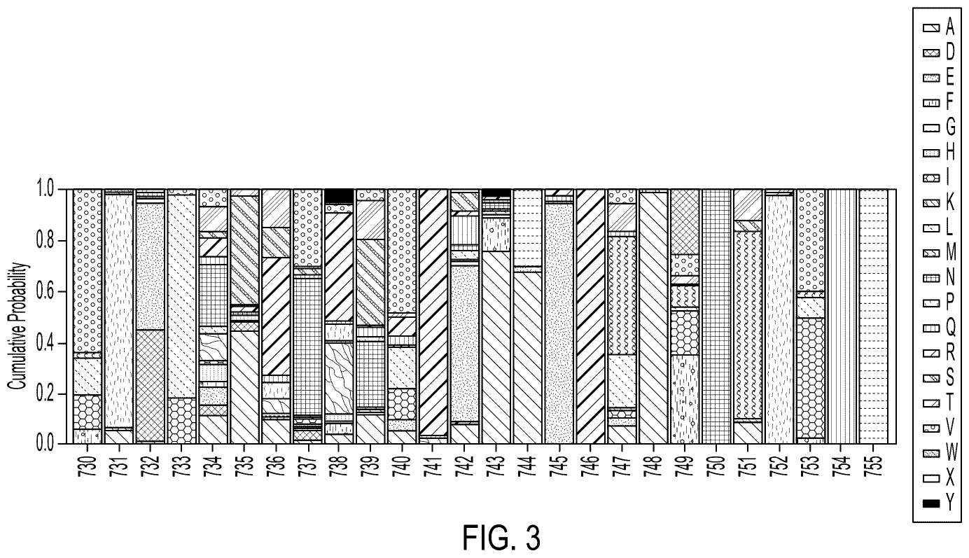

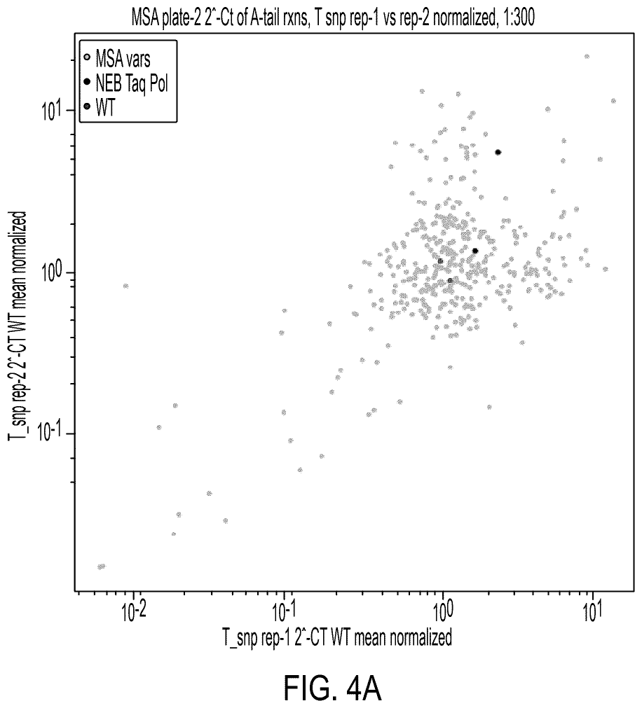

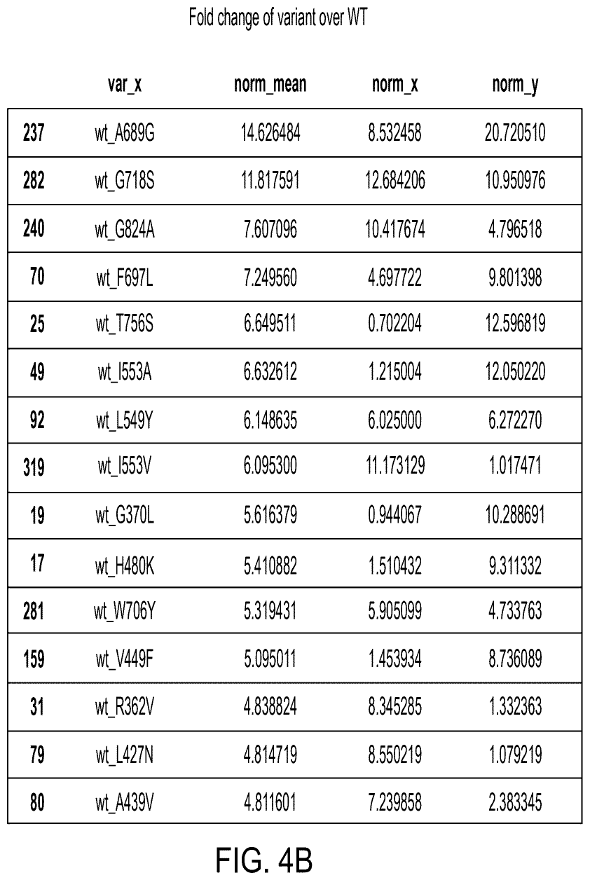

Following the general procedure of Example 1, multiple rounds of optimization/selection were used to generate Taq polymerase variants. Variants from the Taq sequence (SEQ ID NO: 1) were selected based in part on high entropy positions (FIG. 3) and screened using a high throughput qPCR assay (FIGS. 4A-4B). In a first round, single variants were tested for polymerization performance metrics. Multiple sequence alignment (MSA) of a region of Taq Polymerase aligned with sequence homologues of this enzyme. The MSA was performed at a region of the enzyme identified in the literature. Alternative amino acids found in other homologues, but not WT, are the basis of the initial design of TaqIT variants (FIG. 3).

Enzyme variants identified by MSA were assayed using a 384 well plate workflow. Two replicates were performed and the ligation to T-tailed adapters was quantified by qPCR. The scatter plot (FIG. 4A) of activity normalized to the WT, showed the correlation between the two replicates. There is a cloud of variants around the WT, and a subset of variants perform better than WT in one or both replicates. A table of the top variants that performed consistently better than WT across replicates is shown in FIG. 4B.

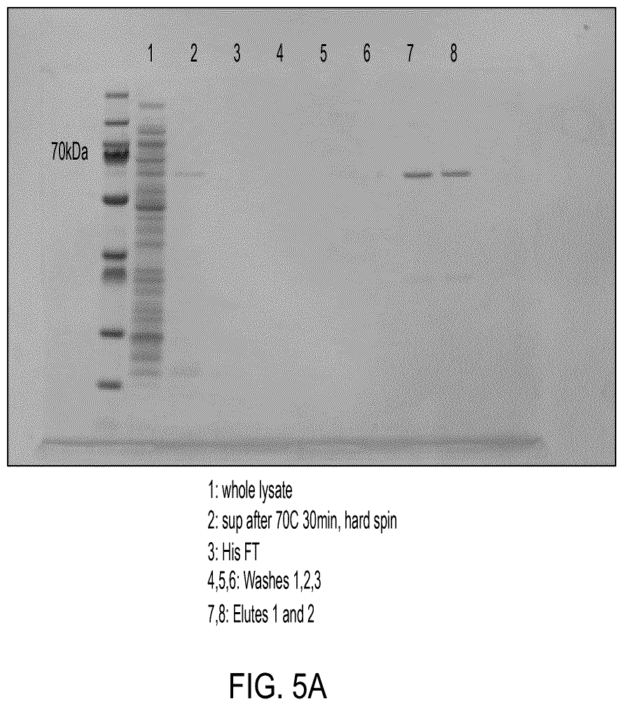

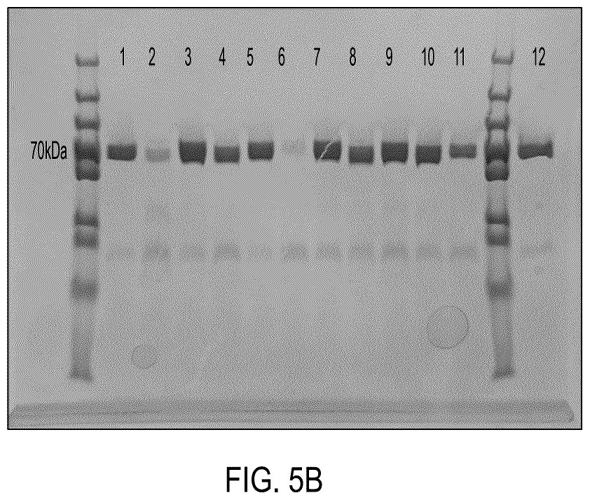

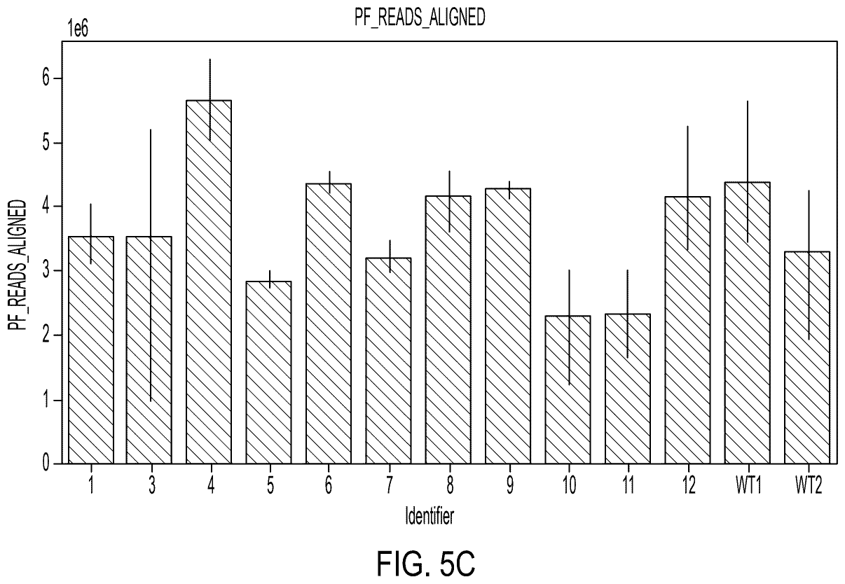

Taq variants were purified by taking advantage of the Taq polymerase heat tolerance. FIG. 5A. Taq variants were expressed as His6-tagged constructs. The His-tagged variants underwent enzymatic lysis (BPER) and heat treatment at 70° C. for 30 minutes. The Taq variant was purified from the heat-stabilized lysate using Ni-NTA column purification for characterization in a next-generation sequencing library preparation assay. The purified variants were quantified by spectrophotometry and purity was evaluated using SDS PAGE. FIG. 5A shows an SDS PAGE gel of purified wild-type TaqIT and FIG. 5B shows an SDS PAGE gel of purified Taq homologues. NGS library preparation was performed using purified TaqIT homologues as the A-tailing enzyme during the end repair, A-tailing reaction. Here the total number of aligned reads is plotted for each enzyme variant (n=3) (FIG. 5C).

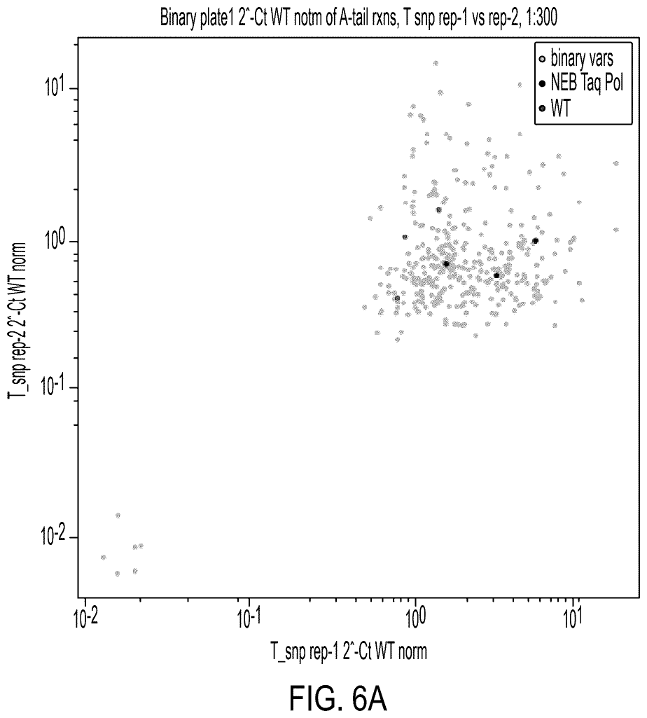

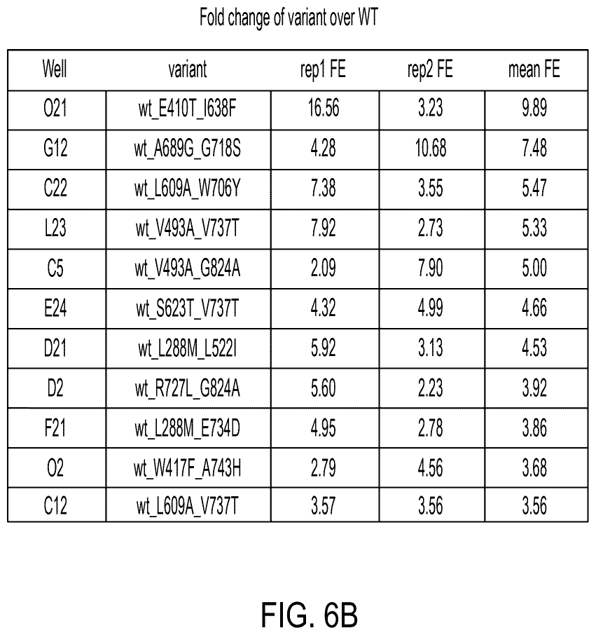

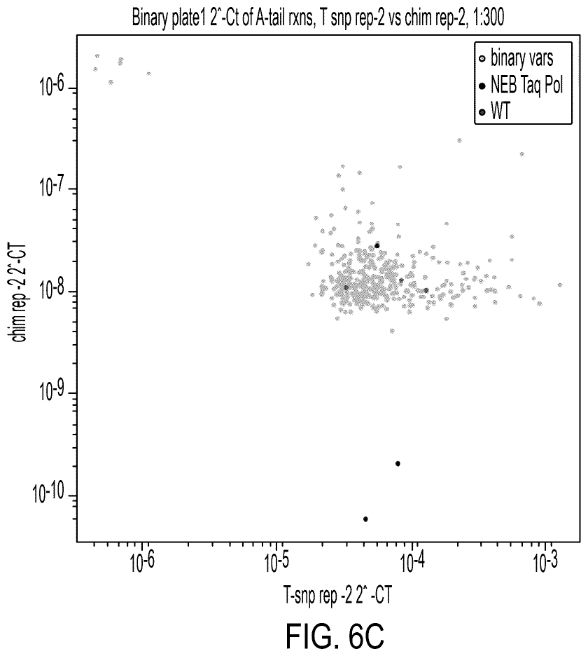

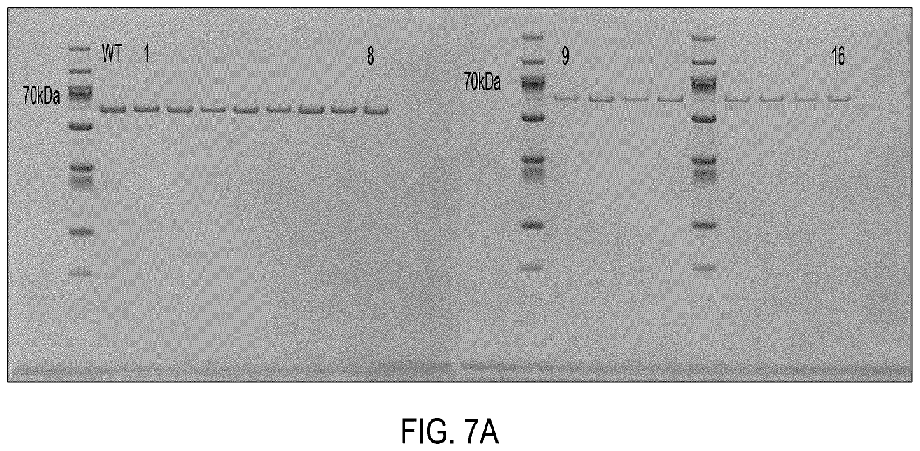

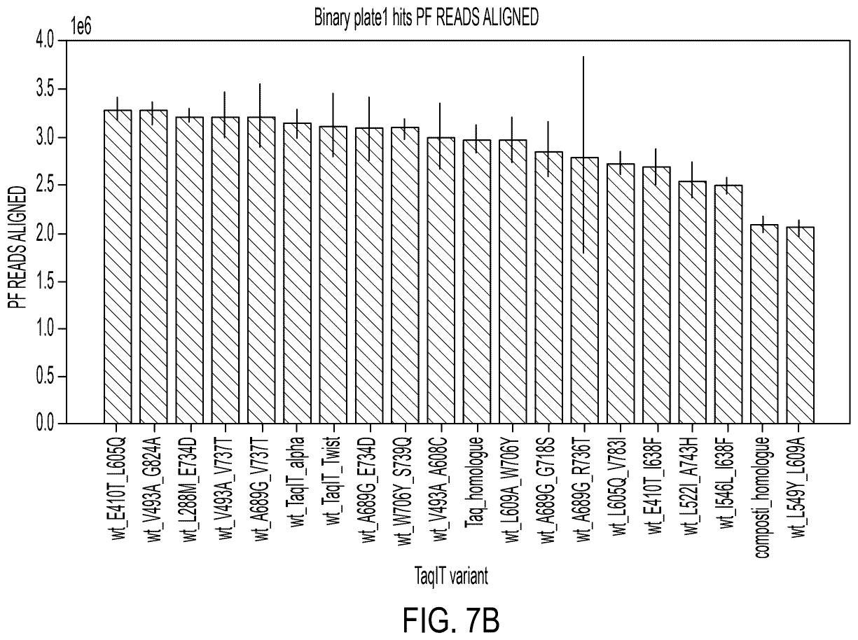

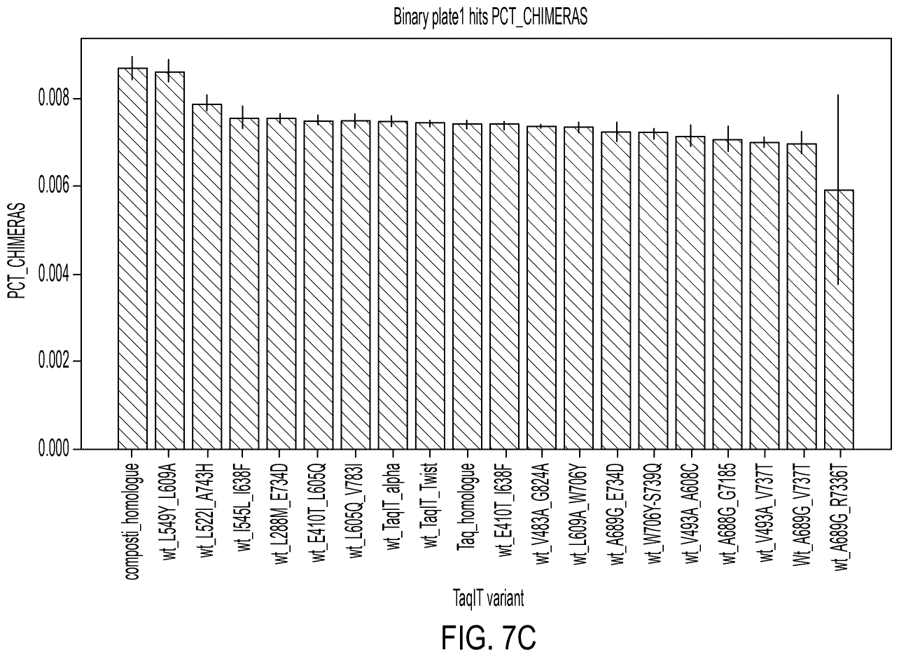

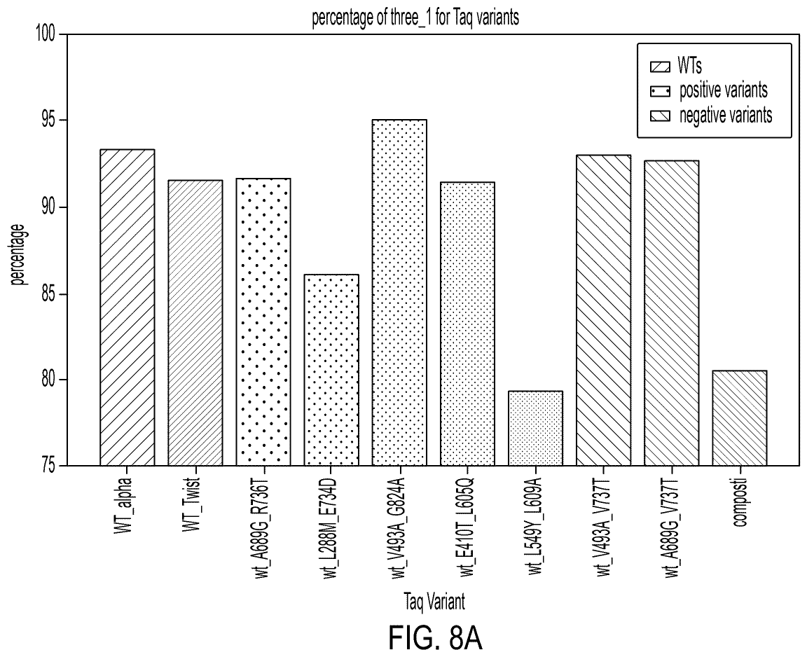

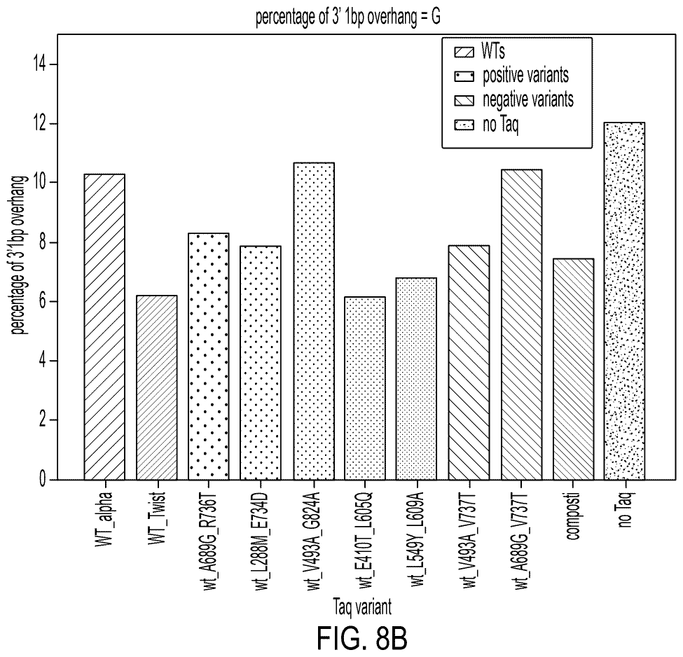

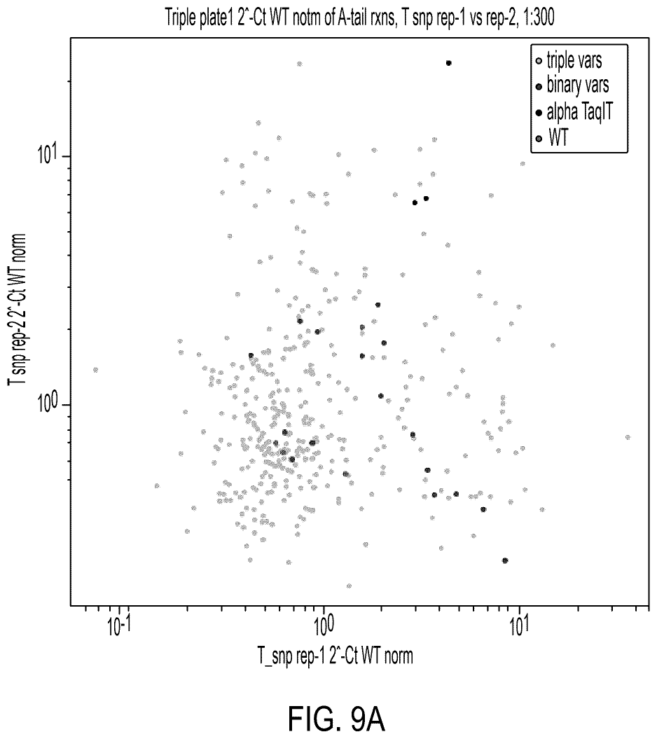

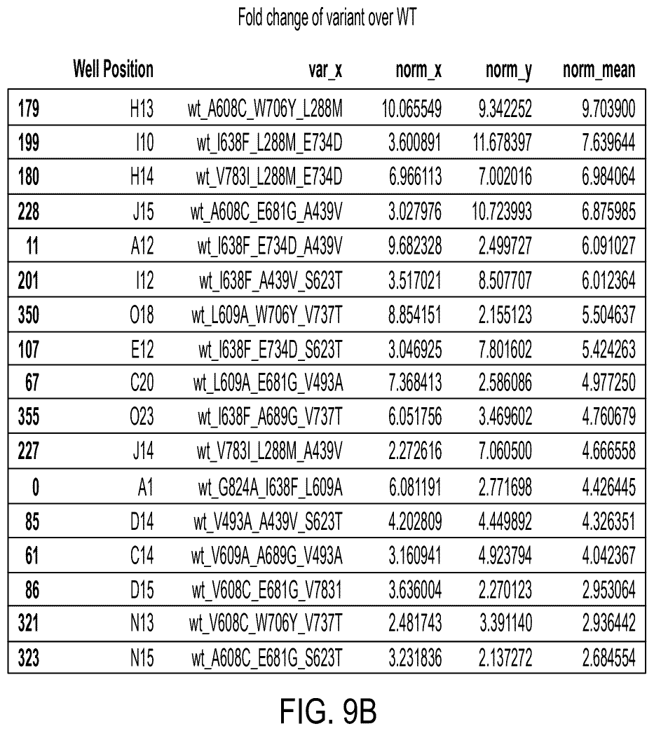

In screening round 2, binary combinations of about 50 single variants were assayed using the 384 well plate workflow above (FIGS. 6A-6C). From this round, winners were selected to be evaluated after purification. The purified variants were quantified by spectrophotometer and purity was evaluated using SDS PAGE (FIG. 7A) prior to being used in NGS library preparation (FIGS. 7B-7C). Enzyme ternary variants were assayed using the 384 well plate workflow above. Two replicates were performed and ligation to the T-tailed adapter was quantified by qPCR. The scatter plot (FIG. 9A) of activity normalized to the WT, shows the correlation between the two replicates. There is a cloud of variants around the WT, and a subset of variants perform better than WT in one or both replicates. A table of the top variants (FIG. 9B) that performed consistently better than WT across replicates (FE=fold enrichment). Binary combinations variants were analyzed for correct tail length and number of 3′ one base pair overhangs with a “G” instead of an “A” position (FIGS. 8A-8B). Using the end composition assay TaqIT binary variants, and one homologue, were evaluated for the percentage of reads that have the desired 1 bp 3′ overhang (FIG. 8A). Using the end composition assay TaqIT binary variants, and one homologue, were evaluated for the percentage of 1 bp 3′ reads that have G tails, an undesired outcome. By decreasing the G-tailing, having higher specificity for A-tailing, the TaqIT variants will create more ligatable molecules for NGS (FIG. 8B).

Binary combinations, with two mutants per sequence, were also constructed SDS-PAGE gel showing a set of purified TaqIT binary variants is shown in FIG. 7A. NGS library preparation was performed using purified TaqIT binary variants as the A-tailing enzyme during the end repair and A-tailing reaction. Here the total number of aligned reads (left) and percent chimera (right) were plotted for each enzyme variant (n=3). (FIGS. 9A-9B). Enzyme tertiary variants were assayed using the 384 well plate workflow above. Two replicates were performed and ligation to T-tailed adapters was quantified by qPCR. The scatter plot (left) of activity normalized to the WT, shows the correlation between the two replicates. There was a cloud of variants around the WT, and a subset of variants perform better than WT in one or both replicates. This plate included a few binary variants from the previous round. Binary variants outperformed WT, and other tertiaries also outperformed some binaries. On the right is a table of the top variants that performed consistently better than WT across replicates. (FIGS. 9A-9B).

In some instances, wild type TaqIT (SEQ ID NO: 2) results in about 8% G tailing (rather than A). For ligation with adapters comprising a T overhang, this may reduce the efficiency of ligation with this type of adapter. Mutants were identified which gave improved A-tailing efficiency and selectivity of no more than 2% G tailing (Table 3).

TABLE 3

| Variant | Mutations (numbering relative to SEQ ID NO: 1) |
|---|---|
| TaqIT_1 | L605C_T664I |
| TaqIT_2 | T664I_D732A |
| TaqIT_3 | T664I_R736Q |
| TaqIT_4 | V493L_T664I |
| TaqIT_5 | L609A_A689G_V493A |
| TaqIT_6 | V449F_T664I |
| TaqIT_7 | L522I_R736K |
| TaqIT_8 | T664I_K738V |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

The present disclosure is further described by the following non-limiting items.

Item 1. A variant polypeptide comprising at least one amino acid mutation relative to SEQ ID NO: 1.

Item 2. The polypeptide of item 1, wherein the polypeptide comprises at least 80% similarity to any one of SEQ ID NOs: 3-9.

Item 3. The polypeptide of item 1, wherein the polypeptide comprises at least 90% similarity to any one of SEQ ID NOs: 3-9.

Item 4. The polypeptide of item 1, wherein the polypeptide comprises at least 95% similarity to any one of SEQ ID NOs: 3-9.

Item 5. The polypeptide of item 1, wherein the polypeptide comprises at least 98% similarity to any one of SEQ ID NOs: 3-9.

Item 6. The polypeptide of item 1, wherein the polypeptide comprises any one of SEQ ID NOs: 3-9.

Item 7. The polypeptide of any one of items 1-6, wherein the mutation comprises one or more of an addition, deletion, and substitution.

Item 8. The polypeptide of any one of items 1-7, wherein the deletion comprises 250-300 amino acids from the N-terminus relative to SEQ ID NO: 1.

Item 9. The polypeptide of any one of items 1-7, wherein the polypeptide comprises at least 2 amino acid mutations relative to SEQ ID NO: 1.

Item 10. The polypeptide of any one of items 1-7, wherein the polypeptide comprises at least 3 amino acid mutations relative to SEQ ID NO: 1.

Item 11. The polypeptide of any one of items 1-7, wherein the polypeptide comprises at least 4 amino acid mutations relative to SEQ ID NO: 1.

Item 12. The polypeptide of any one of items 1-11, wherein the mutations are at one or more of positions V449F, V493L, L522I, L605C, T664I, E681G, W706Y, D732A, R736K, R736Q, and G824A relative to SEQ ID NO: 1.

Item 13. The polypeptide of item 12, wherein the mutations are at two or more of positions V449F, V493L, L5221, L605C, T664I, E681G, W706Y, D732A, R736K, R736Q, and G824A relative to SEQ ID NO: 1.

Item 14. The polypeptide of item 12, wherein the mutations are selected from two or more of V449F, V493L, L522I, L605C, T664I, E681G, W706Y, D732A, R736K, R736Q, and G824A relative to SEQ ID NO: 1.

Item 15. The polypeptide of item 14, wherein the mutations are selected from one or more of V449F, V493L, L522I, L605C, T664I, E681G, W706Y, D732A, R736K, R736Q, and G824A relative to SEQ ID NO: 1.

Item 16. The polypeptides of any one of items 1-15, wherein the polypeptide further comprises a purification tag.

Item 17. A nucleic acid encoding for the polypeptide of any one of items 1-16.

Item 18. A vector comprising the nucleic acid of item 17.

Item 19. The vector of item 18, wherein the vector comprises a plasmid.

Item 20. A cell comprising the nucleic acid of item 17.

Item 21. The cell of item 20, wherein the cell comprises a bacterial cell.

Item 22. A method of expressing the polypeptide of any one of items 1-15.

Item 23. The method of item 22, wherein expression comprises translation of the nucleic acid sequence of any one of items 1-16.

Item 24. The method of item 22 or 23, wherein the method comprises an in vivo method.

Item 25. The method of item 22 or 23, wherein the method comprises a cell-free method.

Item 26. A method for extending a first polynucleotide comprising:

contacting a first polynucleotide with a nucleotide and polypeptide of any one of items 1-16 to form an extended polynucleotide.

Item 27. The method of item 26, wherein the first polynucleotide comprises genomic DNA or a fragment thereof.

Item 28. The method of item 26, wherein the first polynucleotide comprises cDNA.

Item 29. The method of item 26, wherein the nucleotide comprises adenosine triphosphate.

Item 30. The method of any one of items 26-29, wherein the method is selective for incorporation of a single nucleotide.

Item 31. The method of item 30, wherein the method results in at least 90% selectivity for a single nucleotide vs. incorporation of multiple nucleotides.

Item 32. The method of item 30, wherein the method results in at least 95% selectivity for a single nucleotide vs. incorporation of multiple nucleotides.

Item 33. The method of any one of items 26-29, wherein the method is selective for incorporation of a nucleotide type.

Item 34. The method of item 33, wherein the method results in at least 90% selectivity for the nucleotide type.

Item 35. The method of item 33, wherein the method results in at least 95% selectivity for the nucleotide type.

Item 36. The method of item 33, wherein the method results in at least 95% selectivity for A over G.

Item 37. The method of any one of items 26-36, wherein the method further comprises ligating an adapter to the extended polynucleotide.

Item 38. The method of item 37, wherein the adapter comprises a complementary overhang to the extended polynucleotide.

Item 39. The method of item 37, wherein the method further comprises extending a second polynucleotide.

Item 40. The method of item 39, wherein the first polynucleotide and the second polynucleotide are hybridized.

Item 41. A kit for nucleic library preparation comprising:
a ligase;
a polymerase having the sequence of the polypeptide of any one of items 1-16; and
at least one adapter.

Item 42. A method for preparing a sequencing library comprising:
providing a plurality of nucleic acids;
end-repairing the plurality of nucleic acids;
performing a-tailing on the nucleic acids using a polymerase having the sequence of the polypeptide of any one of items 1-16; and
ligating at least one adapter to the nucleic acids using a ligase.

Item 43. The method of item 42, wherein the plurality of nucleic acids is derived from cfDNA.

Item 44. The method of item 42, wherein the plurality of nucleic acids is derived from ctDNA.

1. A polypeptide comprising an amino acid sequence comprising at least one amino acid mutation relative to SEQ ID NO: 1.

2. The polypeptide of claim 1, wherein the polypeptide comprises an amino acid sequence at least 80%, at least 90%, at least 95%, at least 98%, or 100% homologous to any one of SEQ ID NOs: 3-9.

3. The polypeptide of claim 1, wherein the mutation comprises an addition, deletion, substitution, or a combination thereof.

4. The polypeptide of claim 1, wherein the deletion comprises 250-300 amino acids from the N-terminus relative to SEQ ID NO: 1.

5. The polypeptide of any one of the preceding claims, wherein the polypeptide comprises at least 2, at least 3, or at least 4 amino acid mutations relative to SEQ ID NO: 1.

6. The polypeptide of claim 1, wherein the mutations are at one or more of positions V449, V493, L522, L605, T664, E681, W706, D732, R736, R736, and G824 relative to SEQ ID NO: 1.

7. The polypeptide of claim 1, wherein the mutations are selected from one or more of V449F, V493L, L522I, L605C, T664I, E681G, W706Y, D732A, R736K, R736Q, and G824A relative to SEQ ID NO: 1.

8. The polypeptide of claim 1, further comprising a purification tag.

9. A nucleic acid molecule encoding for the polypeptide of claim 1.

10. A vector comprising the nucleic acid of claim 9.

11. A cell comprising the nucleic acid molecule of claim 9.

12. A method for extending a first polynucleotide, the method comprising:
contacting the first polynucleotide with a nucleotide and a polypeptide to form an extended polynucleotide,
wherein the polypeptide comprises an amino acid sequence comprising at least one amino acid mutation relative to SEQ ID NO: 1.

13. The method of claim 12, wherein the first polynucleotide comprises genomic DNA or a fragment thereof, cDNA, or adenosine triphosphate.

14. The method of claim 12, wherein the method is at least 90% selective for incorporation of a single nucleotide.

15. The method of claim 12, wherein the method is at least 90% selective for incorporation of a nucleotide type.

16. The method of claim 15, wherein the method is at least 95% selective for adenine (A) over guanine (G).

17. The method of claim 1, further comprising ligating an adapter to the extended polynucleotide.

18. The method of claim 17, wherein the adapter comprises a complementary overhang to the extended polynucleotide.

19. The method of claim 17, further comprising extending a second polynucleotide, wherein the first polynucleotide and the second polynucleotide are hybridized.

20. A method for preparing a sequencing library comprising:
providing a plurality of nucleic acids;
end-repairing the plurality of nucleic acids;
performing a-tailing on the plurality of nucleic acids using a polymerase, wherein the polymerase comprises an amino acid sequence comprising at least one amino acid mutation relative to SEQ ID NO: 1; and
ligating at least one adapter to the nucleic acids using a ligase.
